(19) United States
(12) Patent Application Publication
(10) Pub. No.: US 2023/0398534 A1
(43) Pub. Date: Dec. 14, 2023

(54) DIAGNOSTIC CARTRIDGE CAPABLE OF AUTOMATIC SOLUTION SUPPLY AND MULTIPLE DETECTION

(71) Applicant: QSTAG CO., LTD., Incheon (KR)

(72) Inventors: Da Woon HAN, Seoul (KR); Eung Kyu PARK, Suwon-si (KR); Khac Phu Cuong TRUONG, Suwon-si (KR); Hye Ri OH, Anyang-si (KR); Ik Soo SHIN, Seoul (KR); Dong Hoon LEE, Suwon-si (KR)

(73) Assignee: QSTAG CO., LTD., Incheon (KR)

(21) Appl. No.: 18/032,302

(22) PCT Filed: Oct. 28, 2021
(86) PCT No.: PCT/KR2021/015312

§ 371 Date: Apr. 17, 2023

(30) Foreign Application Priority Data
Nov. 19, 2020 (KR) 10-2020-0155955
Nov. 19, 2020 (KR) 10-2020-0155956
Nov. 19, 2020 (KR) 10-2020-0155957

(51) Int. Cl. B01L 3/00

(52) U.S. Cl. CPC B01L 3/5023; B01L 2300/0825; B01L 2400/0406; B01L 2300/069

(57) ABSTRACT

The present invention relates to a diagnostic cartridge, which may comprise: a housing that has formed therein a receiving space, and has formed, on the upper surface thereof, a first inlet and a second inlet that are spaced apart from each other in communication with the receiving space and an outer space; and a solution supply device that is integrally or detachably mounted in the second inlet, receives a washing solution and a reaction solution, and sequentially introduces at intervals the washing solution and the reaction solution into a membrane pad through the second inlet.

### TECHNICAL FIELD

The present invention relates to a diagnostic cartridge which is capable of automatic solution supply and multiple detection, and more specifically to a diagnostic cartridge which can improve the convenience of usage and can perform a diagnosis regardless of a diagnostic line and a dispensing distance.

### BACKGROUND ART

Many diagnostic strips have been developed and used to quickly and accurately measure the concentration or presence of an analyte in bodily fluids such as blood and urine, and recently, immunosensor strips, DNA sensor strips and the like based on biospecific binding have been developed.

The diagnostic strip using nanoparticles based on the lateral flow immunoassay method is the most common disposable point-of-care diagnostic strip, and the nanoparticles are aggregated by antigen binding to form bands, and it is possible to visually determine positive/negative. However, when analyzing a low concentration of antigen, there is a limitation in assay sensitivity that occurs in the process of visually observing band formation. Although there is a way to improve the sensitivity so that bands can be visually distinguished at low concentrations, there are limitations in terms of materials. In addition, although there is a method of improving the sensitivity by analyzing an image through fluorescence or light emission, there is a problem in that an additional image analysis device is required, which is not suitable for a disposable point-of-care diagnostic kit. Moreover, although the colored band can be analyzed as a general optical or fluorescent image and the result can be digitized, there is a problem in that an additional device for image analysis is required.

In order to analyze a low-concentration antigen, high-sensitivity electrochemical analysis has been applied to lateral flow immunoassay, and to this end, the quantitative analysis of a reacted substrate through the catalytic role of an enzyme bound to the detection antibody is important, and thus, it is necessarily accompanied by a washing step to remove the same and then a step of dispensing a certain amount of substrate. This process is similar to the enzyme-linked immunosorbent assay (ELISA) that is performed in the laboratory, and there are limitations in the complexity of the analysis process in several steps, making it difficult for general users to use.

Therefore, there has been a need for a diagnostic strip that can be easily used by general users and can be used as a point-of-care diagnostic kit by improving the limitations of sensitivity that occur in the visual observation method.

In addition, Japanese Patent Laid-Open No. 2019-113425 (published on Jul. 11, 2019) proposed a structure in which the sample introduction part is located at the other end of each test line, by providing a plurality of test lines by branching a carrier which is connected to the assembly pad.

However, there has been a problem in that accurate diagnosis was difficult due to the difference in dispensing distances from the assembly pad side to each test line.

### DISCLOSURE

### Technical Problem

In the technical problems to be solved by the present invention, an object of the present invention is to provide a diagnostic strip that can be easily used by general users and can be analyzed quickly and easily in the field.

Another object of the present invention is to provide a diagnostic cartridge which is capable of performing multiple diagnosis regardless of a plurality of diagnostic lines and the dispensing distance.

In addition, still another object of the present invention is to provide a diagnostic cartridge which is capable of increasing the reliability of multiple diagnosis by uniformly flowing a fluid regardless of the dispensing distance by using a capillary phenomenon.

Still another object of the present invention is to provide a diagnostic cartridge which is capable of highly reliable diagnosis by preventing the evaporation of a fluid.

### Technical Solution

The diagnostic cartridge of the present invention may include a housing that has formed therein a receiving space, and has formed, on the upper surface thereof, a first inlet and a second inlet that are spaced apart from each other in communication with the receiving space and an outer space; and a solution supply device that is integrally or detachably mounted in the second inlet, receives a washing solution and a reaction solution, and sequentially introduces at intervals the washing solution and the reaction solution into a membrane pad through the second inlet.

In an exemplary embodiment of the present invention, the solution supply device may be an automatic supply part which is mounted integrally or detachably to the second inlet, receives a washing solution and a reaction solution and introduces the washing solution into the membrane pad when a sliding member moves, and after a predetermined time has elapsed after the washing solution is introduced, the reaction solution is introduced into the second inlet.

In an exemplary embodiment of the present invention, the automatic supply part may include a first housing which is coupled to the second inlet; a first receptacle which is located at an upper part of the first housing and receives the washing solution, and in which the bottom surface is removed by a strip line having one end connected to the bottom surface and the other end connected to the sliding member so as to introduce the washing solution into the second inlet; and a second receptacle which is located at an upper part of the first housing and a side surface of the first receptacle to receive the reaction solution, and is broken by the action of a breaking part to introduce the washing solution into the second inlet.

In an exemplary embodiment of the present invention, the breaking part may be operated after the sliding member slides and a set time has elapsed, and break by puncturing or pressing the second receptacle.

In an exemplary embodiment of the present invention, the solution supply device may include a first housing which is coupled to the second inlet; a first receptacle which is located at an upper part of the first housing, receives the washing solution, and is broken by the action of a breaking part so as to introduce the washing solution into the second inlet; and a second receptacle which is located at an upper part of the first housing and a side surface of the first receptacle to receive the reaction solution, and is broken by the action of the breaking part to introduce the washing solution into the second inlet.

In an exemplary embodiment of the present invention, the breaking part may break by sequentially puncturing or pressing the first receptacle and the second receptacle.

In an exemplary embodiment of the present invention, breaking lines may be formed on the bottom surfaces of the first receptacle and the second receptacle such that the breaking lines are broken by the pressure of the breaking part.

In an exemplary embodiment of the present invention, the diagnostic cartridge may further include an absorbent pad which is disposed in the receiving space between the first inlet and the second inlet and has one side surface in contact with the inner upper surface of the housing and the other side surface spaced apart from the membrane pad, wherein the absorbent pad may be formed by being folded multiple times in different and opposite directions.

In an exemplary embodiment of the present invention, the diagnostic cartridge may further include an absorbent pad which is disposed in the receiving space between the first inlet and the second inlet and has one side surface in contact with the inner upper surface of the housing and the other side surface spaced apart from the membrane pad, wherein the absorbent pad may include an elastic member which is in contact with an inner upper surface of the housing and an absorbent member which is disposed below the elastic member.

In an exemplary embodiment of the present invention, the area of each layer, the number of folds and the volume of the absorbent pad may be adjusted according to the amounts of the washing solution and the reaction solution introduced into the second inlet.

In an exemplary embodiment of the present invention, the diagnostic cartridge may further include a sample pad which is disposed in the receiving space to be in contact with at least one surface of the membrane pad and absorbs the sample solution introduced through the first inlet; and a sliding member which includes a gripping part for gripping the sample pad and an extension part which is formed to extend in a horizontal direction from the gripping part and has at least a portion protruding outward through a side surface of the housing, wherein the sliding member slides in a horizontal direction such that the sample pad is spaced apart from the membrane pad, and the other side surface of the absorbent pad is in contact with the membrane pad, and wherein the gripping part includes an upper plate and a lower plate that are spaced apart vertically to form a separation space in which the sample pad is accommodated, and includes a through-hole which penetrates an upper surface of the upper plate and communicates with the sample pad and the first inlet, and the gripping part forms a bending part in which both ends of the upper plate are bent toward a lower part.

In an exemplary embodiment of the present invention, the gripping part may include an absorbent pad receptacle which extends upward from an upper surface of the upper plate and has the other end bent in a direction opposite to the extension part to accommodate the absorbent pad therein.

In an exemplary embodiment of the present invention, the membrane pad may be pre-treated with a capture antibody that specifically binds to a predetermined antigen on one side surface.

In an exemplary embodiment of the present invention, the absorbent pad receptacle may form an inclined surface with an end portion inclined toward the lower part, and the absorbent pad may move along the inclined surface when the sliding member slides and be seated on the upper surface of the membrane pad.

In an exemplary embodiment of the present invention, the diagnostic cartridge may further include an electrode part which is located between the second inlet and the absorbent pad and is closely disposed on the upper surface of the membrane pad.

In an exemplary embodiment of the present invention, the diagnostic cartridge may further include a pressing member which is formed to extend downward from the inner upper surface of the housing to face the electrode part and presses an electrochemical sensor unit.

In an exemplary embodiment of the present invention, the diagnostic cartridge may further include a first discoloration checker and a second discoloration checker which are formed to penetrate the upper surface between the first inlet and the second inlet to confirm discoloration of the membrane pad.

In an exemplary embodiment of the present invention, the housing may include a code display unit for displaying a code which is capable of reading information through a device on an outer upper surface.

In an exemplary embodiment of the present invention, the diagnostic cartridge may further include an indentation part which is formed to extend from the inner upper surface of the housing to the lower part between the first inlet and the second inlet to form a space into which the absorbent pad is inserted, and in which the lower side can be opened and closed by the sliding member.

In an exemplary embodiment of the present invention, the diagnostic cartridge may include a membrane pad which is disposed in the receiving space such that both ends of the housing are exposed to a first inlet and a second inlet of the housing, and includes a plurality of diagnostic lines; and a cutting part for cutting such that a plurality of the diagnostic lines are divided, respectively.

In an exemplary embodiment of the present invention, the membrane pad may include a first pad which is exposed to the outside through the first inlet; a second pad which is exposed to the outside through the second inlet; a plurality of diagnostic lines for connecting the first pad and the second pad; and reaction parts that are located in each of the diagnostic lines.

In an exemplary embodiment of the present invention, the second pad may be provided in plurality, and the second pad may be connected to the first pad by the same number of diagnostic lines as the second pad.

In an exemplary embodiment of the present invention, the diagnostic cartridge may further include a first space forming part and a second space forming part for forming a microcavity between the diagnostic lines at the upper part and the side surface of the diagnostic lines that are adjacent to the first pad and the second pad.

In an exemplary embodiment of the present invention, each of the first space forming part and the second space forming part may have a hydrophilic coating layer formed on a surface facing the diagnosis line.

In an exemplary embodiment of the present invention, the diagnostic cartridge may further include a sliding member for removing the first space forming part on the membrane pad.

In an exemplary embodiment of the present invention, a part of the sliding member may support a lower side of the absorbent pad, remove the first space forming part, and move the absorbent pad downward such that a part of the absorbent pad comes into contact with the membrane pad.

In an exemplary embodiment of the present invention, the diagnostic cartridge may further include a liquid impermeable sheet which covers part or all of the diagnostic line.

### Advantageous Effects

The present invention has the effect of automating the introduction of each solution such that general users can easily use the same.

In addition, the present invention has the effect of increasing the reliability of multiple diagnosis by uniformly flowing a fluid regardless of the dispensing distance by using a capillary phenomenon.

Moreover, the present invention has the effect of increasing the reliability of diagnosis by providing a structure for preventing the evaporation of a fluid.

### DESCRIPTION OF DRAWINGS

FIG. 1 is a perspective view of a diagnostic strip according to the first example of the present invention.

FIG. 2 is an exploded perspective view of FIG. 1.

FIG. 3 is a cross-sectional view taken along line A-A′ of the diagnostic strip of FIG. 1.

FIG. 4 is a block diagram of a solution supply device applied to the present invention.

FIG. 5 is a view illustrating an absorbent pad of the diagnostic strip of FIG. 1.

FIGS. 6 to 11 are operational diagrams illustrating the operation process of a diagnostic strip according to an exemplary embodiment of the present invention.

FIG. 12 is an exemplary view of the configuration for breaking a solution supply device 70 of the present invention.

FIG. 13 is a configuration diagram of an exemplary embodiment of the first receptacle.

FIG. 14 is a perspective view of a diagnostic strip according to the second example of the present invention.

FIG. 15 is a cross-sectional view taken along line B-B′ of the diagnostic strip of FIG. 14.

FIGS. 16 to 20 are operational diagrams illustrating the operation process of a diagnostic strip according to the second example of the present invention.

FIG. 21 is a usage status diagram of the diagnostic strip according to the second example of the present invention.

FIG. 22 is a cross-sectional view of a diagnostic strip according to the third example of the present invention.

FIGS. 23 to 27 are operational diagrams illustrating the operation process of a diagnostic strip according to the third example of the present invention.

FIG. 28 is a perspective view of a diagnostic strip according to the fourth example of the present invention.

FIG. 29 is an exploded perspective view of FIG. 28.

FIG. 30 is a cross-sectional view taken along line A-A′ of the diagnostic strip of FIG. 28;

FIG. 31 is a block diagram of an automatic supply part according to the fourth example of the present invention.

FIGS. 32 to 36 are operational diagrams illustrating the operation process of a diagnostic strip according to the fourth example of the present invention.

FIG. 37 is an exemplary diagram of the configuration for breaking the automatic supply part.

FIG. 38 is a cross-sectional configuration diagram of a diagnostic strip according to the fifth example of the present invention.

FIG. 39 is a cross-sectional configuration diagram of a diagnostic strip according to the sixth example of the present invention.

FIG. 40 is a cross-sectional configuration diagram of a diagnostic cartridge according to the seventh example of the present invention.

FIG. 41 is a plan view of the main part of FIG. 40.

FIG. 42 is a partial perspective view of FIG. 40.

FIG. 43 is a cross-sectional view taken along line A-A in FIG. 42.

FIG. 44 is a cross-sectional view of a state in which the sliding member is moved.

FIG. 45 is a cross-sectional view of a state in which the membrane pad is cut by the cutting part.

FIG. 46 is a plan view of the membrane pad in the cut state of FIG. 45.

FIG. 47 is a plan view of a membrane pad according to another exemplary embodiment of the present invention.

- Explanation of Reference Numerals -

| | |
|---|---|
| 1: Diagnostic strip | 10: Housing |
| 11: Receiving space | 12: First inlet |
| 13: Second inlet | 14: Arrival confirmation port |
| 16: First discoloration checker | |
| 17: Second discoloration checker | |
| 18: Code display unit | 19: Indentation part |
| 20: Membrane pad | 30: Sample pad |
| 40: Absorbent pad | 50: Sliding member |
| 60: Electrode part | |
| 70: Solution supply device, automatic | |
| supply part | |

### MODES OF THE INVENTION

Advantages and characteristics of the present invention, and methods for achieving the same will become apparent from the following exemplary embodiments taken in conjunction with the accompanying drawings. However, the present invention is not limited to the disclosed exemplary embodiments, but may be implemented in various different ways. The exemplary embodiments are provided to complete the disclosure of the present invention and to allow a person having ordinary skill in the art to which the present invention pertains to fully understand the scope of the present invention, and the present invention is only defined by the claims.

Like reference numerals refer to like components throughout the specification.

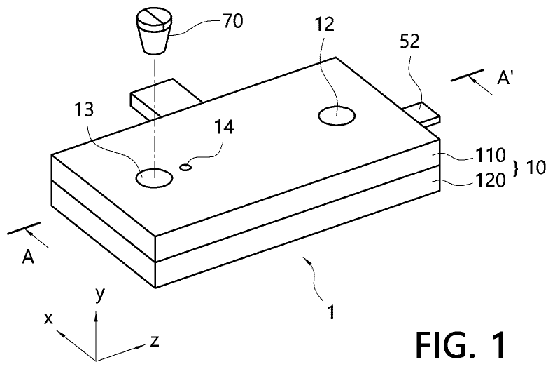

Hereinafter, the diagnostic strip according to the first example of the present invention will be described with reference to FIGS. 1 to 11.

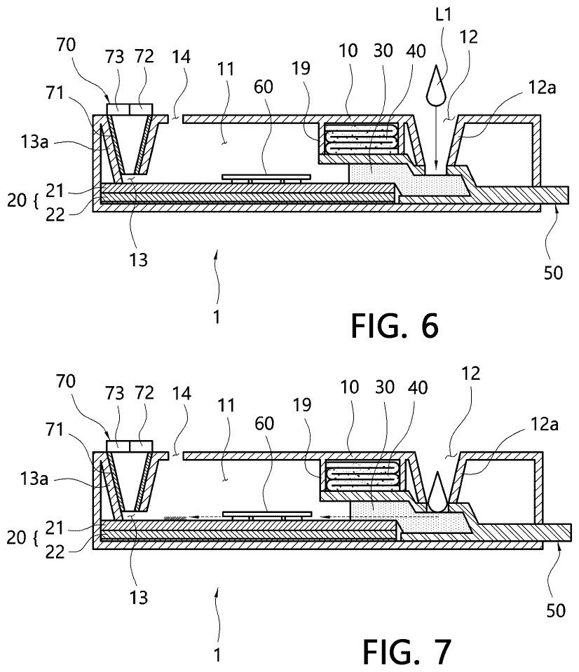

After the configuration of the diagnostic strip according to the first example of the present invention will be described in detail with reference to FIGS. 1 to 5, the operation process will be described with reference to FIGS. 6 to 11.

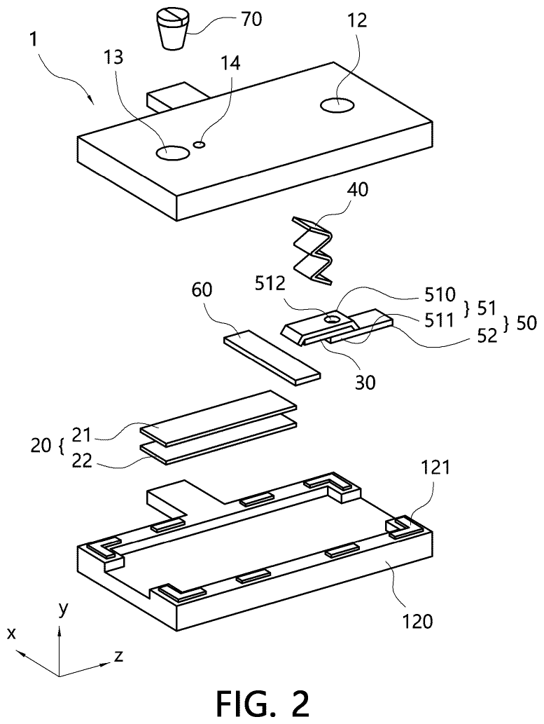

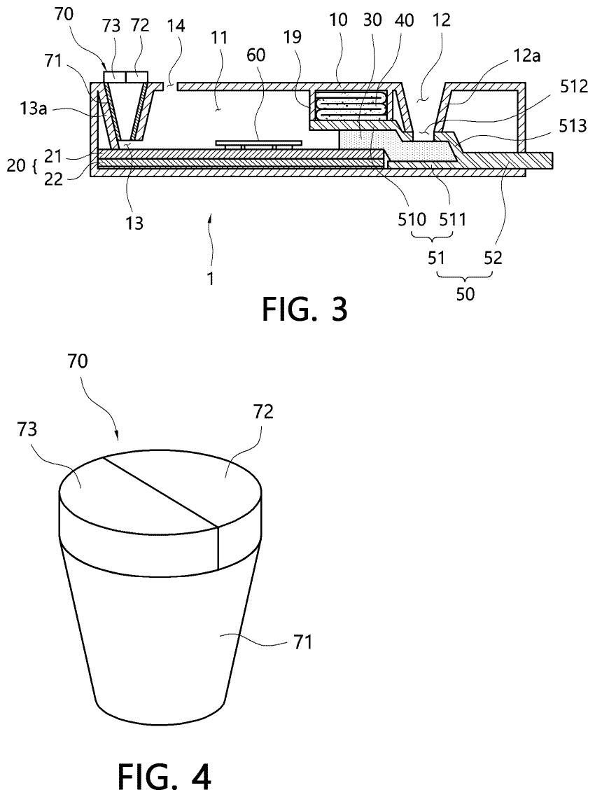

FIG. 1 is a perspective view of a diagnostic strip according to the first example of the present invention, FIG. 2 is an exploded perspective view of FIG. 1, FIG. 3 is a cross-sectional view taken along line A-A′ of the diagnostic strip of FIG. 1, FIG. 4 is a block diagram of a solution supply device applied to the present invention, and FIG. is a view illustrating an absorbent pad of the diagnostic strip of FIG. 1.

Referring to FIGS. 1 to 5, respectively, the diagnostic strip 1 of the present invention is a diagnostic kit for allowing general users to conveniently measure the concentration or presence of an analyte in bodily fluids such as blood and urine, and to quickly and accurately analyze in the field.

The diagnostic strip 1 of the present invention includes a housing 10, a membrane pad 20, a sample pad 30, an absorbent pad 40 and a sliding member 50 which are located inside the housing 10. Specifically, the diagnostic strip 1 of the present invention is formed with a receiving space 11 therein, and includes a housing for forming a first inlet 12 and a second inlet 13 that are disposed to be spaced apart from each other on the upper surface to communicate between the receiving space 11 and the outer space, a membrane pad 20 which is exposed to the second inlet 13 and disposed in the receiving space 11, a sample pad 30 which is disposed in the receiving space 11 to be in contact with at least one surface of the membrane pad 20 and absorbs a sample solution L1 which is introduced into the first inlet 12, an absorbent pad 40 which is disposed between the first inlet 12 and the second inlet 13 and has one side surface in contact with the inner upper surface of the housing 10 and the other side surface spaced apart from the membrane pad 20, a gripping part 51 for gripping the sample pad, a sliding member 50 which includes an extension part 52 that is formed to extend from the gripping part 51 in a horizontal direction and has at least a portion thereof protruding outward by penetrating a side surface of the housing 10, and a solution supply device 70 which is capable of sequentially supplying a washing solution W1 and a reaction solution L2 by being integrally or detachably coupled to the second inlet 13, wherein the sliding member 50 slides in a horizontal direction such that the sample pad 30 is spaced apart from the membrane pad 20, and the other surface of the absorbent pad 40 contacts the membrane pad 20.

The housing 10 forms the main body of the diagnostic strip 1, and it may be formed in a rectangular parallelepiped shape to form a receiving space 11 therein. As illustrated in the drawing, although it is described as an example that the housing 10 is formed to extend in the z-axis direction and a cross-section cut in a horizontal line is formed in a rectangular shape, the present invention is not limited thereto, and a cross-sectional shape cut by a horizontal line may be formed in an elliptical shape rather than a rectangular shape. The housing 10 accommodates the membrane pad 20, the sample pad 30, the absorbent pad 40 and the sliding member 50 in the receiving space 11. The housing 10 may be separately formed vertically such that each component accommodated in the receiving space 11 can be easily disposed, and may include a first member 110 and a second member 120 that are formed separately to be coupled. The first member 110 and the second member 120 form the upper and lower portions of the housing 10 that are separated up and down, respectively, and they may include an upper surface and a lower surface of the housing 10, respectively, and may be separated and coupled to each other by coupling grooves (not illustrated) and protrusions 121 that are formed on surfaces facing each other. In the first member 110 including the upper surface of the housing 10, a first inlet 12 and a second inlet 13 communicating with the receiving space 11 and the outer space are formed to be spaced apart from each other, and an arrival confirmation port 14 may be formed between the first inlet 12 and the second inlet 13. The first inlet 12 and the second inlet 13 are through-holes for introducing the sample solution L1, the washing solution W1 and the reaction solution L2 into the receiving space 11, respectively, and they may be formed to penetrate the upper and lower surfaces of the first member 110 to be spaced apart along the z-axis, which is the longitudinal direction of the housing 10.

A solution supply device 70 may be coupled or integrally fixed to the second inlet 13. The solution supply device 70 includes a cylindrical housing 71 for coupling with the second inlet 13, a first receptacle 72 which is located at an upper part of the housing 71 to receive the washing solution W1, and a second receptacle 73 which is located at an upper part of the housing 71 and on the side surface of the first receptacle 72 to receive the reaction solution L2.

Although the exemplary embodiment of the present invention is described as including a cylindrical housing 71, the first receptacle 72 and the second receptacle 73 may be installed to be in direct contact with a second guide surface 13a of the second inlet 13.

As will be described in detail below, the first receptacle 72 and the second receptacle 73 may introduce the received washing solution W1 and reaction solution L2 through the second inlet 13 by methods such as breakage, fracture, cutting, pressing and the like, respectively, in the course of an analysis process at appropriate timing.

That is, by breaking the first receptacle 72 and injecting the washing solution W1 to proceed with a washing step, the second receptacle 73 is broken and the reaction solution L2 is injected to analyze the reactivity of HRP, and the washing solution W1 and the reaction solution L2 may be injected into the second inlet in stages.

In the present specification, examples of the washing solution W1 may include a washing buffer partially containing a surfactant such as tween and the like, and examples of the reaction solution L2 may include a solution containing a substrate that reacts with an enzyme (peroxidase, phosphatase, etc.) bound to the detection antibody. The sample solution L1, the washing solution W1 and the reaction solution L2 may be introduced to be opposite to each other through the first inlet 12 and the second inlet 13. The first inlet 12 and the second inlet 13 have a feature that can minimize non-specific reactions by implementing the input directions in which the sample solution L1, the washing solution W1 and the reaction solution L2 are introduced in opposite directions. The arrival confirmation port 14 is a through-hole for confirming that the sample solution L1 introduced into the first inlet 12 has reached a specific position along the membrane pad 20, and it may be formed to penetrate the upper and lower surfaces of the first member 110, and it may be formed in parallel between the first inlet 12 and the second inlet 13 along the z-axis. The arrival confirmation port 14 may be formed to be spaced apart from the first inlet 12 and the second inlet 13, and be formed at a position adjacent to the second inlet 13 than the first inlet 12.

Meanwhile, the housing 10 may include a first guide surface 12a which extends from an inner upper surface, a second guide surface 13a and an indentation part 19.

When the present invention is described in detail with reference to FIG. 3, the housing 10 may include a first guide surface 12a which is formed to extend downward from the lower surface of the first member 110, a second guide surface 13a, an indentation part 19 and a pressing member (not illustrated).

The first guide surface 12a and the second guide surface 13a for guiding the movement of the sample solution L1, the washing solution W1 and the reaction solution L2 that are introduced into the first inlet 12 and the second inlet 13, and they may be formed to extend downward from the first inlet 12 and the second inlet 13 toward the receiving space 11. The first guide surface 12a and the second guide surface 13a may be formed to have a diameter of an end portion smaller than diameters of the first inlet 12 and the second inlet 13 to be inclined downward. The first guide surface 12a and the second guide surface 13a may be formed to be inclined toward the lower portion, so as to guide the sample solution L1, the washing solution W1 and the reaction solution L2 that are introduced into the first inlet 12 and the second inlet 13 to be introduced into specific positions of the sample pad 30 and the membrane pad 20. In addition, a portion of the first guide surface 12a may be formed to have a longer length than other portions such that the membrane pad 20 can be closely fixed to the upper surface of the second member 120.

The indentation part 19 forms a space in which the absorbent pad 40 to be described below is inserted and guides the movement of the absorbent pad 40, and it may be formed to extend downward from the inner upper surface of the housing 10. The indentation part 19 may be formed to extend downward from the lower surface of the first member 110 between the first inlet 12 and the second inlet 13.

The indentation part 19 may be disposed to be closer to the first inlet 12 between the arrival confirmation port 14 and the first inlet 12 to form a space into which the absorbent pad 40 is inserted. The indentation part 19 accommodates the absorbent pad 40 in the inner space, and the opened lower side can be opened and closed by the sliding member 50, which will be described below in detail in the operation process.

A pressing member is for pressing the electrode part 60 which is disposed in the receiving space 11, and it may be formed to extend downward from the inner upper surface of the housing 10. The pressing member may be formed to extend downward from the lower surface of the first member 110 between the first inlet 12 and the second inlet 13. The pressing member may be formed at a position facing the electrode part 60 between the arrival confirmation port 14 and the indentation part 19 to press the electrode part 60.

The membrane pad 20 is disposed in the receiving space 11 of the housing 10 including the first member 110 and the second member 120.

The membrane pad 20 is disposed in the inner receiving space 11 of the housing 10.

The membrane pad 20 may be disposed on the upper surface of the second member 120 and may be disposed to be exposed by the second inlet 13. The membrane pad 20 may include a nitrocellulose (NC) pad 21, and it may be formed to extend along the longitudinal direction of the housing 10 so as to face the second inlet 13, the arrival confirmation port 14, the press member and the indentation part 19. At least a portion of the membrane pad 20 is pre-treated with a capture antibody that specifically binds to a specific target antigen, and the detection antibody and capture material that secondarily bind to the antigen bound to the capture antibody are not limited to the antibody, and they may be composed of proteins, carbohydrates, nucleotides, aptamers and the like.

The membrane pad 20 may be formed by combining a cover which is capable of reducing the interaction between the fluids in the nitrocellulose pad 21 and the second member 120, and for example, it may be formed by binding the nitrocellulose pad 21 and polyethylene terephthalate (PET) 22. The membrane pad 20 may be formed of a member that minimizes interaction, such as a corrosion reaction by a fluid, and it may be a member having an adhesive formed thereon.

One end portion of the membrane pad 20 may absorb the sample solution L1 that is introduced from the first inlet 12 from the sample pad 30, and when the sample solution L1 moves in the longitudinal direction to reach the arrival confirmation port 14, it may absorb the washing solution W1 and the reaction solution L2 that are introduced from the other end portion into the second inlet 13. The reaction solution absorbed by the membrane pad 20 is transferred to the electrode part 60, and the electrode part 60 may generate an electric signal, which will be described in detail during the operation process. The membrane pad 20 is characterized in that one side surface is pre-treated with a capture antibody that specifically binds to a predetermined antigen. The membrane pad 20 is not limited to the detection antibody and the capture material that binds secondarily to the antigen bound to the capture antibody, and it may be composed of proteins, carbohydrates, nucleotides, aptamers and the like. The membrane pad 20 may be described as an example in which capture is coated in advance on one side surface, and the capture and detector may not be limited to materials of the antibody.

The membrane pad 20 may quantitatively analyze the reaction that occurs on the surface through the electrode part 60 by electrochemical analysis. Meanwhile, the electrode part 60 is disposed on the upper surface of the membrane pad 20, and the sample pad 30 which is in contact with and separated from the membrane pad 20 is disposed on one side of the membrane pad 20.

The electrode part 60 obtains a signal from a reaction in which the reaction solution that is introduced into the second inlet 13 and absorbed by the membrane pad is oxidized/reduced by an enzyme of the detection antibody, and it may be disposed to be in close contact with the upper surface of the membrane pad 20 which is located between the second inlet 13 and the absorbent pad 40. The electrode part 60 includes a substrate on which electrodes are patterned, and two electrodes or three electrodes may be patterned. For example, the electrode part 60 includes a counter electrode, a working electrode and a reference electrode, and it may be disposed on the upper surface of the membrane pad 20 to face the pressing member. Such an electrode part 60 reacts with the solution that is introduced into the first inlet 12 and the second inlet 13 to cause an oxidation/reduction reaction to analyze the concentration or presence of an analyte contained in the solution.

Meanwhile, the diagnostic strip 1 according to the first example of the present invention is described as an example of measuring the concentration of an antigen by oxidation/reduction reaction, but the present invention is not limited thereto, and the presence or concentration of an analyte may be analyzed by confirming that the membrane pad 40 is discolored without forming the electrode part 60. In order to analyze the signal of the presence or absence of binding of the detection antibody, the enzymes used in combination with the detection antibody are peroxidase and phosphatase, and typically, horseradish peroxidase and alkaline phosphatase may be used. As a substrate thereof, TMB, dianisidine, phenylenediamine, NBT/BCIP and pNPP may be used for electrochemical and discoloration reactions, and lumino, CSPD, 1,2-dioxetane and the like may be used for luminescence reaction. In addition, fluorescence may be analyzed by attaching a fluorescent dye to the detection antibody.

The sample pad 30 is a pad that absorbs the sample solution L1 that is introduced into the first inlet 12. The sample pad 30 may be disposed under the first guide surface 12a so as to be exposed through the first inlet 12, and it may be disposed in contact with at least one surface of the membrane pad 20 in the receiving space 11 so as to absorb the sample solution L1 that is introduced from the first inlet 12 to transfer to the membrane pad 20. As illustrated in FIG. 3, the lower surface of the sample pad 30 may contact the upper surface and one side surface of the membrane pad 20 at the same time, but the present invention is not limited thereto, and it may be disposed in the receiving space 11 such that one side surface of the end portion is in contact with one side surface of the end portion of the membrane pad 20, or at least a portion thereof is in contact therewith. The sample pad 30 disposed in this way may be slidably moved in a horizontal direction, which is the longitudinal direction of the housing 10, by the sliding member 50 such that it can be contacted and separated from the membrane pad 20.

The sliding member 50 is for contacting and separating the sample pad 30 from the membrane pad 20, and it is formed of a soft material and disposed in the receiving space 11. The sliding pad includes a gripping part 51 for gripping the sample pad 30, and an extension part 52 which extends horizontally from the gripping part 51 such that at least a portion thereof penetrates the side surface of the housing 10 and is disposed to protrude to the outside, and the sample pad 30 may be in contact with and separated from the membrane pad 20 while sliding in the horizontal direction by pulling the extension part 52 that is disposed to protrude from the outside of the housing 10.

The gripping part 51 is for gripping the sample pad 30, and it may include an upper plate 510 and a lower plate 511 that are vertically spaced apart from each other. The gripping part 51 is formed in a ‘C’ shape to form a separation space in which the sample pad 30 is accommodated between the upper plate 510 and the lower plate 511, and it may be formed with a through-hole 512 that penetrates the upper surface of the upper plate 510 to communicate with the separation space and the first inlet 12. The gripping part 51 forms a through-hole 512 communicating with the first inlet 12 such that the sample pad 30 which is disposed in the separation space may absorb the sample solution L1 that is introduced into the first inlet through the through-hole 512. The gripping part 51 may form a bending part 513 in which an end portion of the upper plate 510 is bent downward. The bending part 513 is for preventing the sample pad from being spaced apart from the separation space when the sliding member 50 slides.

The extension part 52 forms a handle for moving the gripping part 51, and it is formed to extend from the end portion of the gripping part 51 in the horizontal direction. The extension part 52 is formed to extend along the −z-axis direction from the gripping part 51, and the end portion penetrates the side surface of the housing 10 and is disposed to protrude outside the housing 10. At least a portion of the extension part 52 is disposed on the outside of the housing 10, and the user pulls the end portion of the extension part 52 to slide the sliding pad in the z-axis and −z-axis directions such that the sample pad 30 may be in contact with and separated from the membrane pad 20.

Meanwhile, although the sliding pad is described as an example of sliding in the horizontal direction and the extension part 52 is formed to extend in the z-axis and −z-axis directions and slides along the longitudinal direction of the housing 10, the present invention is not limited thereto, and the sample pad 30 may contact with and be separated from the membrane pad 20 while being slidingly formed to extend in the x-axis or −x-axis direction.

Subsequently, the gripping part 51 may be formed such that the upper plate 510 is longer than the lower plate 511. The gripping part 51 may be positioned below the indentation part 19 when the upper plate 510 is formed to be longer than the lower plate 511 and the sliding pad slides in the z-axis direction. The upper plate 510 of the gripping part 51 is positioned below the indentation part 19 while the sliding pad slides in the z-axis direction and the −z-axis direction, or it is positioned such that it is completely deviated from the lower side of the indentation part 19 so as to open or close the lower side of the indentation part 19. In this way, the sliding pad opens and closes the open lower side of the indentation part 19 such that the absorbent pad 40 accommodated in the indentation part 19 is completely accommodated inside the indentation part 19, or at least a portion may be separated from the indentation part 19 and contact the membrane pad 20. This will be described below in detail through the operation process after the absorbent pad 40 is described.

The absorbent pad 40 is for absorbing the washing solution W1 that is introduced through the second inlet 13 and absorbed into the membrane pad 20, and it is formed of a material that expands in volume when the solution is absorbed. For example, the absorbent pad 40 may be formed of a material such as glass fiber, cotton and the like, and it may be disposed between the first inlet 12 and the second inlet 13.

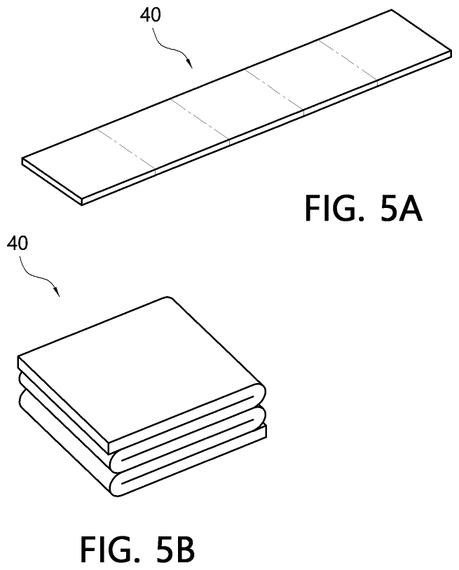

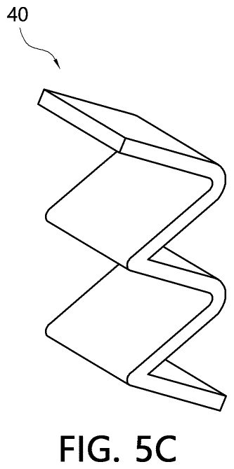

The absorbent pad 40 may adjust the thickness, material, volume and the like, according to the amount of the washing solution W1 that is introduced through the second inlet 13 while the first receptacle 72 of the solution supply device 70 is broken. Referring to FIG. 5a, the absorbent pad 40 may be formed of a thin pad which is capable of absorbing a solution. The absorbent pad 40 may be formed by being cut to a size that can be inserted into the indentation part 19, and the cut and formed thin pad may be formed by folding in different directions multiple times as illustrated in FIG. 5b. The absorbent pad 40 is described as an example that it is folded to form five layers as shown in the drawings, but the present invention is not limited thereto, and the area, volume, number of folds, thickness, material and the like of each layer may be adjusted according to the amount of the washing solution that is introduced into the second inlet 13.

The absorbent pad 40 may be folded multiple times in opposite directions to form a stacked structure, and one side surface thereof is in contact with the inner upper surface of the housing 10, and the other side surface thereof is disposed to be spaced apart from the membrane pad 20. The absorbent pad 40 may have different elasticity depending on the area, volume, thickness, number of times of folding, material and the like. When pressure is applied up and down after folding the absorbent pad 40 multiple times as in FIG. 5b, each layer may be stacked as in FIG. 5c, and when no pressure is applied to the upper or lower surface of the absorbent pad 40, each layer may be spaced apart and open up and down as shown in FIG. 5b. The absorbent pad according to the first example of the present invention may be compressed and restored by elasticity and have a structure which is capable of absorbing a solution, but the present invention is not limited thereto, and it may be formed such that an elastic member that can be compressed and restored and an absorbent member that absorbs the solution may be formed separately and closely disposed or bonded, and this will be described in detail through the diagnostic strip 1a of the second example.

The absorbent pad 40 is inserted into the indentation part 19 such that the lower side of the indentation part 19 is closed by the sliding member 50 to be spaced apart from the membrane pad 20, and when the sliding member 50 slides and the lower side of the indented portion 19 is opened, a portion thereof may be separated from the indentation part 19 due to elasticity and may come into contact with the upper surface of the membrane pad 20 on the other side surface.

That is, before the sliding member 50 slides, the gripping part 51 closes the lower side of the indentation part 19 such that the absorbent pad 40 is completely inserted into the indentation part 19, and thus, it may be spaced apart from the membrane pad 20, the sample pad 30 may be disposed to be in contact with the membrane pad 20, and the through-hole 512 may be disposed to communicate with the first inlet 12.

In addition, when the extension part 52 of the sliding member 50 slides outwardly of the housing 10, the gripping part 51 opens the lower side of the indentation part 19 such that the other side surface of the absorbent pad 40 is in contact with the membrane pad 20, the sample pad 30 moves away from the membrane pad 20, and the upper surface of the gripping part 51 can close the end of the first guide surface 12a, and it will be described in more detail with reference to the operation process.

Hereinafter, the operation process of a diagnostic strip according to the first example of the present invention will be described with reference to FIGS. 6 to 11.

Although the drawing illustrates that the first receptacle 72 and the second receptacle 73 protrude to the outside of the housing 10, they may be in a retracted form.

FIGS. 6 to 11 are operational diagrams illustrating the operation process of a diagnostic strip according to the first example of the present invention.

Referring to FIG. 6, FIG. 6 is a diagram illustrating that the sample solution L1 is introduced into the first inlet 12. For the sample solution L1, a detection antibody-horseradish peroxide (HRP) solution that has been pre-bound with the antigen sample to be analyzed is used. Peroxidase enzymes such as HRP can oxidize specific substrates, and the degree of this enzyme-substrate oxidation/reduction reaction can be measured through optical measurement methods such as discoloration, luminescence, fluorescence analysis and the like, and in addition, the amount of antigen can be quantitatively measured through the high-sensitivity electrochemical measurement methods and the like. Accordingly, the sample solution L1 in which the detection antibody and HRP are combined is introduced into the first inlet 12. In this case, the sample pad 30 must be exposed through the first inlet 12, while the sample pad 30 is exposed through the first inlet 12 and the sample pad 30 is in contact with the membrane pad 20. The sample solution L1 that is introduced into the first inlet 12 may be absorbed by the sample pad 30.

Referring to FIG. 7, FIG. 7 is a diagram illustrating that the sample solution L1 that is introduced into the first inlet 12 falls on the sample pad 30, and the sample pad 30 absorbs the sample solution L1 such that the membrane pad 20 which is in contact with the sample pad 30 is becoming wet.

The sample pad 30 absorbs the sample solution L1 that is introduced from the first inlet 12, and at least a portion thereof is in contact with the membrane pad 20 such that the absorbed sample solution L1 is transferred to the membrane pad 20. The membrane pad 20 may absorb the sample solution L1 from one end that is in contact with the sample pad 30, and the sample solution L1 absorbed from the sample pad 30 may become wet from one end portion to the other end portion. It can be confirmed that the sample solution L1 becomes wet from one end portion of the membrane pad through the sample pad 30 to the other end portion through the electrode part 60. In this case, it can be confirmed that the sample solution L1 is absorbed from the arrival confirmation port 14 to the other end portion of the membrane pad 20, and when the sample solution L1 reaches the lower side portion of the arrival confirmation port 14, the sample pad 30 is separated from the membrane pad 20.

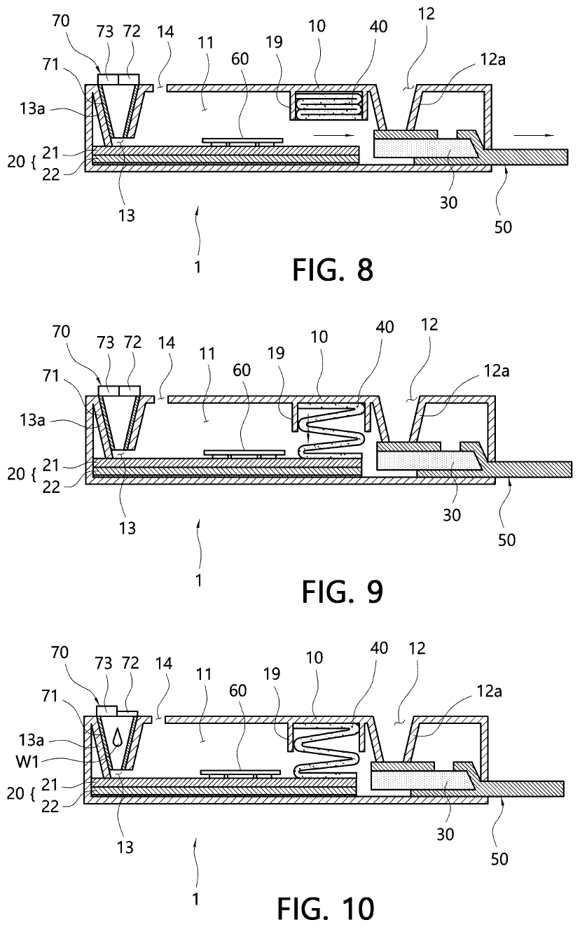

Referring to FIG. 8, FIG. 8 is a diagram illustrating that the sliding pad slides such that the sample pad 30 and the membrane pad 20 are separated. When the sample solution L1 is absorbed by the membrane pad 20 and reaches the lower side of the arrival confirmation port 14, the extension part 52 of the sliding pad is pulled from the outside of the housing 10. When the extension part 52 is pulled, the gripping part 51 which is integrally formed with the extension part 52 may slide and move, and at the same time, the sample pad 30 is separated from the membrane pad 20. When the gripping part 51 slides while the sliding pad is pulled to the outside of the housing 10, the sample pad 30 is separated from the membrane pad 20, and the upper plate 510 of the gripping part 51 may open the lower side of the indentation part 19. As the lower side of the indentation part 19 is opened, the absorbent pad 40 which is inserted and disposed in the indentation part 19 may at least partially protrude from the indentation part 19 by elasticity.

Referring to FIG. 9, FIG. 9 is a diagram illustrating that a portion of the absorbent pad 40 protrudes outwardly of the indentation part 19 due to elasticity. When the lower side of the indentation part 19 is opened, the absorbent pad 40 may protrude to the outside of the indentation part 19 by elasticity, and the other end portion may contact the upper surface of the membrane pad 20. One end portion of the absorbent pad 40 may be in contact with the inner upper surface of the indentation part 19 due to elasticity, and the other end portion may be disposed so as to be in contact with the upper surface of the membrane pad 20. In this case, one end portion of the absorbent pad 40 may be fixed to the inner upper surface of the indentation part 19, but it may not be fixed and may be in close contact by applying a force to the inner upper surface of the indentation part 19 by elasticity.

Next, referring to FIG. 10, FIG. 10 is a diagram illustrating that the washing solution W1 is introduced into the second inlet 13. While the membrane pad 20 is in contact with the absorbent pad 40 and separated from the sample pad 30, the first receptacle 72 is broken, and the washing solution W1 which is accommodated in the first receptacle 72 may be introduced into the second inlet 13.

The material of the first receptacle 72 may be vinyl or resin material, and the washing solution W1 which is accommodated may be leaked by applying pressure manually or automatically or by breaking a part or all of the bottom surface, so as to be introduced through the second inlet 13.

Examples of breaking the first receptacle 72 will be described below in more detail.

The washing solution is introduced into the receiving space 11 through the second inlet 13, falls on the upper surface of the membrane pad 20, is absorbed along the longitudinal direction of the membrane pad 20, and comes into contact with the electrode part 60, and the remainder may be absorbed by the absorbent pad 40.

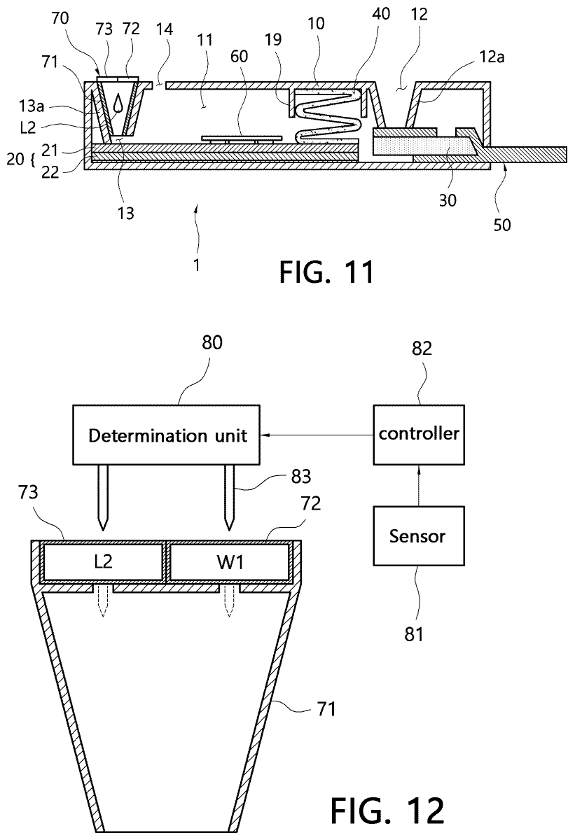

Next, as illustrated in FIG. 11, the second receptacle 73 is broken to supply the reaction solution L2 to the receiving space 11 through the second inlet 13.

In this case, the electrode part 60 may induce an electrochemical reaction of the reaction solution. TMB is oxidized by the HRP enzyme to generate TMB radicals, hydrogen ions and electrons, and the process of reducing TMB that is oxidized according to Formula (2) by an electrochemical reaction at the working electrode is cyclically repeated. By using this principle, the current difference at which the maximum instantaneous current for the oxidation of TMB occurs may be checked by cyclic voltammetry, and the antigen concentration may be measured by the amperometric method. Accordingly, there is an advantage in that an amplified signal can be obtained even with a low concentration of antigen while the oxidation reaction by the enzyme and the reduction reaction by the electrode are continuously cycled.

FIG. 12 is an exemplary view of the configuration for breaking a solution supply device 70 of the present invention.

Referring to FIG. 12, a breaking part 80 for sequentially breaking each of the first receptacle 72 and the second receptacle 73 of the solution supply device 70 may be provided. The breaking part 80 may break a sensor 81 for detecting the position of the extension part 52, and may be operated by a controller 82 that outputs an operation control signal after a set time elapses after the sensor 81 detects the movement of the extension part 52 to break the first receptacle 72 and the second receptacle 73. After the first receptacle 72 is broken, it also has a predetermined delay time until the second receptacle 73 is broken.

The housing 71 has a partition wall formed between the lower ends of the first receptacle 72 and the second receptacle 73 such that the washing solution W1 and the reaction solution L2 do not mix with each other, and this can be applied to the entire described drawings.

In this case, the breaking part 80 may be installed on the diagnostic strip, provided on an analysis device using the diagnostic strip or separately provided outside.

The breaking part 80 includes two awls, and the first receptacle 72 and the second receptacle 73 are respectively punctured by the movement of awls such that the washing solution W1 and the reaction solution L2 that are respectively accommodated in the first receptacle 72 and the second receptacle 73 may be introduced into the second inlet 13 below by gravity.

In the above example, although the breaking part 80 for forming perforations in the first receptacle 72 and the second receptacle 73 by using the awl 83 has been described, examples of breaking the first receptacle 72 and the second receptacle 73 may be implemented in various ways.

For example, the bottom surface of each of the first receptacle 72 and the second receptacle 73 may be broken with a time difference by using a sliding cutter.

The cutter may be of a rotary type.

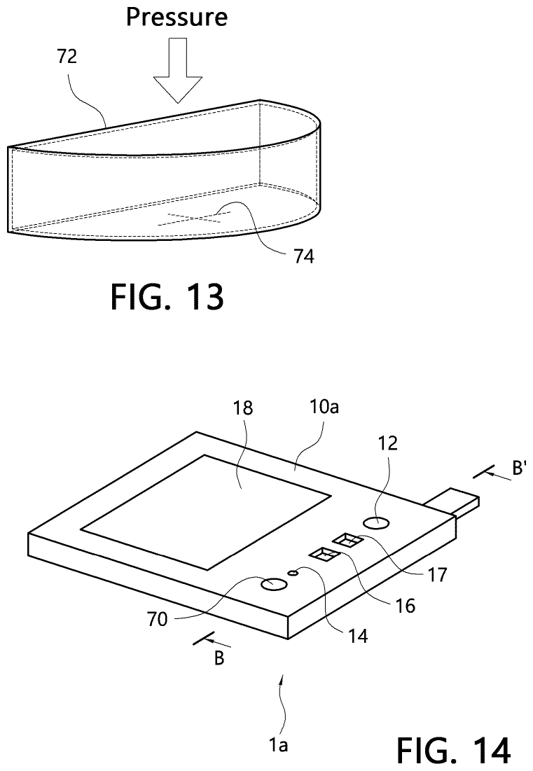

FIG. 13 is a configuration diagram of another exemplary embodiment of the first receptacle 72.

Referring to FIG. 13, the bottom surface of the first receptacle 72 may be formed with a breaking line 74 that is weaker in strength than other parts, and the breaking part 80 may press the upper portion of the first receptacle 72 such that the breaking line 74 is broken, and thus, the washing solution W1 accommodated therein may be introduced into the second inlet 13.

As such, the present invention can supply the washing solution W1 and the reaction solution L2 at a set time through a wide variety of means, thereby improving the convenience of use.

Hereinafter, the diagnostic strip according to the second example of the present invention will be described with reference to FIGS. 14 to 21.

The diagnostic strip 1 according to the second example of the present invention is substantially the same as the first example described above except for the housing 10a and the absorbent pad 40a. Accordingly, the same reference numerals are assigned to the components already described, and the detailed descriptions thereof will be omitted.

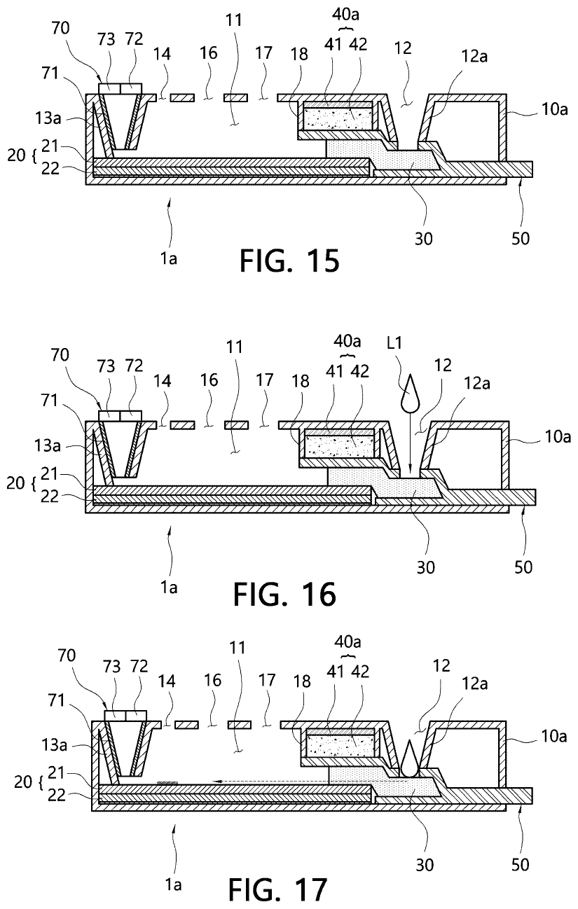

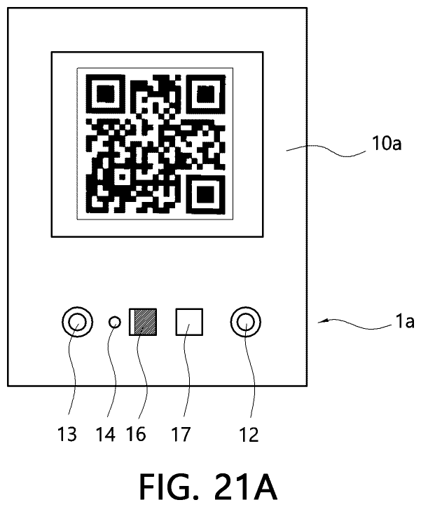

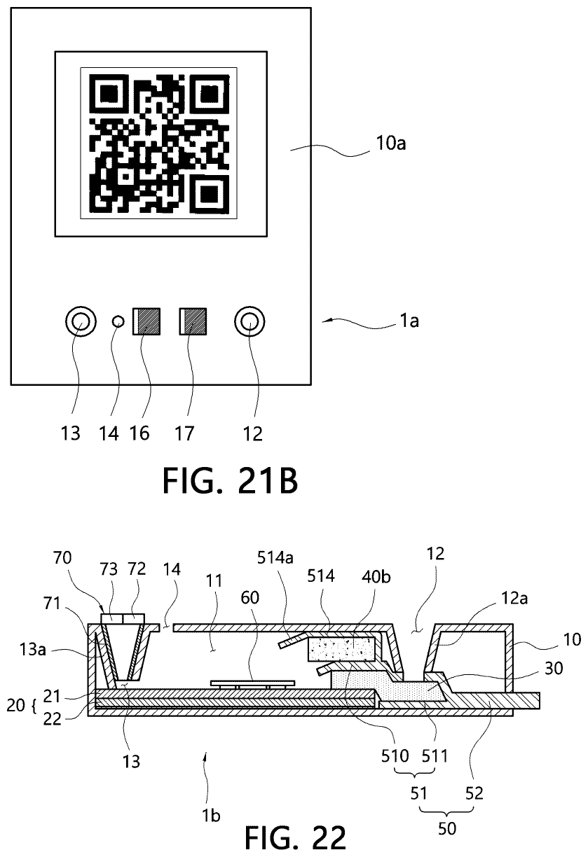

The configuration of the second example of the present invention will be described in detail with reference to FIGS. 14 and 15, and after the operation process will be described with reference to FIGS. 16 to 20, the usage state of the diagnostic strip according to the second example will be described with reference to FIG. 21.

FIG. 14 is a perspective view of a diagnostic strip according to the second example of the present invention, and FIG. 15 is a cross-sectional view taken along line B-B′ of the diagnostic strip of FIG. 14.

Referring to FIGS. 14 and 15, the diagnostic strip 1a according to the second example of the present invention is formed with a first discoloration checker 16, a second discoloration checker 17 and a code display unit 18 in the housing 10a, and an absorbent pad 40a in which an elastic member 41 and an absorbent member 42 are combined is disposed inside the housing 10a.

The housing 10a includes a first discoloration checker 16 and a second discoloration checker 17 that are formed to penetrate the upper surface between the first inlet 12 and the second inlet 13.

The first discoloration checker 16 and the second discoloration checker 17 are through-holes for confirming the discoloration of the membrane pad 20, which are formed to penetrate the upper and lower surfaces of the first member 110a between the first inlet 12 and the second inlet 13 such that at least a portion of the membrane pad may be exposed to the outside of the housing 10a. The first discoloration checker 16 and the second discoloration checker 17 may be formed to be spaced apart from the first member 110 of the housing 10a by a predetermined interval, and may be formed to be spaced apart between the second inlet 13 and the indentation part 19 in which the absorbent pad 40a is disposed. The membrane pad 20 may include one or more reaction parts that are coated with a capture antibody to induce a reaction at positions corresponding to the first discoloration checker 16 and the second discoloration checker 17. Since the reaction part is pre-treated with the capture antibody, the antigen-detecting antibody-enzyme complex included in the sample solution L1 may be bound thereto. Thereafter, when the reaction solution is introduced, the substrate included in the reaction solution may undergo an oxidation reaction by the bound enzyme, and as a result, a luminescence or fluorescence reaction may occur or the color may change.

The solution supply device 70 of the exemplary embodiment described above is applied to the second inlet 13. In this case, the solution supply device 70 is also broken by the breaking part 80 in various ways such that the washing solution W1 and the reaction solution L2 can be sequentially supplied.

The reaction part includes a reagent that changes color by reacting with the detection antibody included in the sample solution L1 in the fluid, and the degree of reaction such as color development, luminescence and fluorescence may vary depending on the concentration of antigen in the sample solution L1, and the degree of color development may vary depending on the concentration of antigen. It is possible to check whether the reaction part is discolored through the first discoloration checker 16 and the second discoloration checker 17 to determine whether it is positive or negative.

A code display unit 18 is formed on the outer upper surface of the housing 10a.

The code display unit 18 may display a code that can read information through a device, such as a QR code (Quick Response code), a bar code and the like, on the outer upper surface of the housing 10. The code display unit 18 may be printed and formed on the upper surface of the first pad or may be manufactured and attached in the form of a sticker, and it may be used for the purpose of checking the diagnosis result of the diagnostic strip 1a by using a camera provided in the user's portable terminal (not illustrated) and an application stored in the portable terminal. However, the code display unit 18 is not applied only to the diagnostic strip 1a of the second example, but may be applied to the housing 10 of the diagnostic strip 1 in the first example so as to be utilized to confirm the concentration and detection of an analyte that are measured by the electrode part 60.

The absorbent pad 40a is inserted into the indentation part 19, and when the indentation part 19 is opened by the movement of the sliding member 50, it is identical to the first example in that it comes into contact with the membrane pad 20 and absorbs the washing solution, but it is slightly different from the absorbent pad 40a in the first example in that it includes an elastic member 41 and an absorbent member 42.

The absorbent pad 40a includes an elastic member 41 which is in contact with the inner upper surface of the housing 10a and an absorbent member 42 which is disposed below the elastic member 41. The elastic member 41 is made of an elastically deformable material, and may include, for example, rubber, a spring and the like. One side surface of the elastic member 41 may be disposed to be in close contact with the inner upper surface of the housing 10a, and the absorption member 42 may be disposed to be in close contact with the other side surface thereof. The elastic member 41 is inserted and disposed inside the indentation part 19, and is compressed and restored as the sliding member 50 opens and closes the lower side of the indentation part 19, so as to push the absorption member 42 which is disposed on the other side surface outside of the indentation part 19. The absorbent member 42 may be made of a material that expands in volume when it absorbs liquid, such as glass fiber, cotton and the like, which can absorb liquid, and one side surface thereof may be disposed to be in close contact with the elastic member 41. When the elastic member 41 is compressed, the absorbent member 42 is inserted into the indentation part 19 together with the elastic member 41, and when the upper plate 510 of the gripping part 51 is disposed on the lower side of the indentation part 19, it may be disposed on the inner side of the indentation part 19 together with the elastic member. Moreover, when the sliding member 50 slides and the lower side of the indentation part 19 is opened, it is pushed out of the indentation part 19 by the restoring force of the elastic member 41, and the end portion thereof may be in contact with the upper surface of the membrane pad 20.

The elastic member 41 and the absorbent member 42 may be integrally formed or formed separately and disposed such that one side surface thereof is in contact with each other, or one side surface thereof may be disposed to be fixed to each other.

Meanwhile, in the diagnostic strip 1a according to the second example, when the indentation part 19 is formed on the inner surface of the housing 10a and the lower side of the indentation part 19 is opened, the indentation part 19 may guide the movement of the absorbent pad 40a. However, the present invention is not limited thereto, and the indentation part 19 may not be necessarily formed.

The diagnostic strip 1a in the second example of the present invention will be described as an example that the absorbent pad 40a includes an elastic member 41 and an absorbent member 42. However, the absorbent pad 40a including the elastic member 41 and the absorbent member 42 is not limited to being applied to the diagnostic strip 1a of the second example, and the absorbent pad 40a may also be applied to the diagnostic strip 1 of the first example, and the diagnostic strip in the third example to be described below.

Moreover, the housing 10a in which the first discoloration checker 16, the second discoloration checker 17 and the code display unit 18 of the present invention are formed, and the absorbent pad 40a in which the elastic member 41 and the absorbing member 42 are coupled may be applied to the diagnostic strip 1 in the first example and the diagnostic strip 1b in the third example.

Hereinafter, the operation process of the diagnostic strip according to the second example of the present invention will be described with reference to FIGS. 16 to 20.

FIGS. 16 to 20 are operational diagrams illustrating the operation process of a diagnostic strip according to the second example of the present invention.

Referring to FIG. 16, it illustrates a diagram for introducing a sample solution L1 into the first inlet 12. As for the sample solution L1, as described in the first example, a solution obtained by pre-reacting an antigen with an enzyme-bound detection antibody is introduced into the first inlet 12. The sample solution L1 which is introduced into the first inlet 12 may be absorbed into the sample pad 30.

Referring to FIG. 17, it is a diagram illustrating that the sample solution L1 which is introduced into the first inlet 12 falls on the sample pad 30, and the sample pad 30 absorbs the sample solution L1 such that the membrane pad 20 becomes wet from one end portion that is in contact with the sample pad 30. The sample pad 30 absorbs the sample solution L1 which is introduced from the first inlet 12, and at least a portion thereof is in contact with the membrane pad 20 such that the absorbed sample solution L1 is transferred to the membrane pad 20. It can be confirmed that the sample solution L1 becomes wet from one end portion to the other end portion of the membrane pad 20 through the sample pad 30, and it can be seen that it reaches the lower portion of the arrival confirmation port 14 from the arrival confirmation port 14. When the sample solution L1 is confirmed from the arrival confirmation port 14, the sample pad 30 is separated from the membrane pad 20.

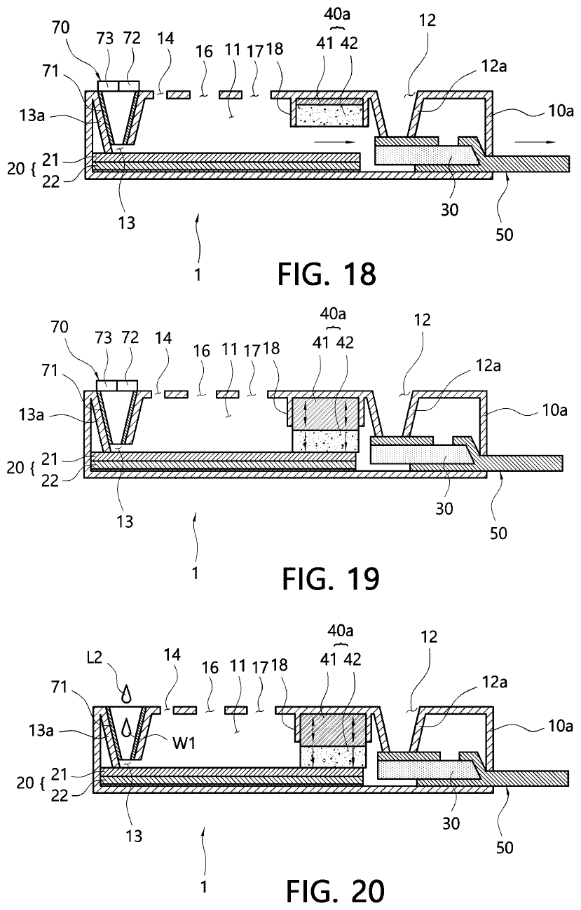

Referring to FIG. 18, it illustrates a diagram of sliding the sliding pad such that the sample pad 30 and the membrane pad 20 are separated. When the sample solution L1 is absorbed by the membrane pad 20 and reaches the lower side of the arrival confirmation port 14, the extension part 52 of the sliding pad is pulled from the outside of the housing 10. When the extension part 52 is pulled, the gripping part 51 which is integrally formed with the extension part 52 may slide and move, and at the same time, the sample pad 30 is separated from the membrane pad 20. When the gripping part 51 slides while the sliding pad is pulled to the outside of the housing 10a, the sample pad 30 is separated from the membrane pad 20, and the upper plate 510 of the gripping part 51 may open the lower side of the indentation part 19. As the lower side of the indentation part 19 is opened, the absorbent pad 40a which is inserted and disposed in the indentation part 19 may allow the absorbent member 42 to protrude from the indentation part 19 by elasticity.

Referring to FIG. 19, it is a diagram illustrating that the absorbent member 42 protrudes outwardly from the indentation part 19 and contacts the membrane pad 20. When the lower side of the indentation part 19 is opened, the absorbent member 42 is pushed out of the indentation part 19 by the restoring force of the elastic member 41 such that the lower side surface of the absorbent member 42 comes into contact with the upper surface of the membrane pad 20. When the absorbent member 42 is in contact with the upper surface of the membrane pad 20, the washing solution W1 and the reaction solution L2 may be sequentially introduced into the second inlet 13.

In FIG. 20, the configuration of the solution supply device 70 is omitted to avoid the duplication of description, but the washing solution W1 and the reaction solution L2 may be supplied from the solution supply device 70. The washing solution W1 is a washing buffer partially including a surfactant such as tween and the like, and it falls on the upper surface of the membrane pad 20 through the second inlet 13 and is absorbed along the longitudinal direction of the membrane pad 20 such that it comes into contact with a reagent pad, and the remaining solution may be absorbed by the absorbent pad 40a. Further, in a portion of the membrane pad 20 that is exposed to the first discoloration checker 16 and the second discoloration checker 17 by the supply of the reaction solution L2, an enzyme of the detection antibody included in the sample solution L1. and a substrate included in the reaction solution react, and as a result, it shows color development, luminescence and fluorescence reactions, and through this, it is possible to immediately check whether the user is negative or positive.

FIG. 21 is a usage status diagram of a diagnostic strip 1a according to the second example of the present invention, in which it can be confirmed that the membrane pad 20 is discolored in the first discoloration checker 16 and the second discoloration checker 17. As shown in FIG. 21a, when color change is confirmed in at least one of the first discoloration checker 16 and the second discoloration checker 17, it can be known that the result is a negative result, and as shown in FIG. 21b, when color change is confirmed in both of the first discoloration checker 16 and the second discoloration checker 17, it can be seen that the result is positive. In addition, the QR code of the code display unit 18 may be photographed through the portable terminal, and a more accurate diagnosis result of the diagnostic strip 1a may be checked by using an application stored in the portable terminal. In this way, the diagnostic strip 1a of the present invention may quickly and accurately confirm a diagnostic result.

Hereinafter, the diagnostic strip according to the third example of the present invention will be described with reference to FIGS. 22 to 27.

The diagnostic strip 1b according to the third example of the present invention is substantially the same as the first example described above except for the sliding member 50a. Accordingly, the same reference numerals are assigned to the components already described, and the detailed descriptions thereof will be omitted.

FIG. 22 is a cross-sectional view of a diagnostic strip according to the third example of the present invention.

Referring to FIG. 22, in the diagnostic strip 1b according to the third example of the present invention, the sliding member 50a includes a gripping part 51 and an extension part 52, and it may further include an absorbent pad receptacle 514 which is formed to extend from the gripping part 51.

The absorbent pad receptacle 514 is for separating and contacting the absorbent pad 40b from the membrane pad 20, and it may be integrally formed with the sliding member 50a. The absorbent pad receptacle 514 is formed to extend upwardly from the upper surface of the upper plate 510 of the gripping part 51, and the other end portion is formed to be bent in a direction opposite to the extension part 52 so as to receive the absorbent pad 40b therein. In other words, one end portion of the absorbent pad receptacle 514 is fixed to the upper surface of the upper plate 510, and it may be formed to extend upward from one end, and the other end portion may be formed to refract in a direction opposite to the direction in which the extension part 52 is formed. The absorbent pad receptacle 514 may be formed in a ‘L’ shape on the upper surface of the upper plate 510, and an inclined surface 514a having an end portion inclined toward the bottom may be formed. The absorbent pad 40b is inserted into the inner space of the absorbent pad receptacle 514 according to whether the sliding member 50a slides and disposed to be spaced apart from the membrane pad 20 or moves along the inclined surface 514a to be separated from the inner space of the absorbent pad receptacle 514 so as to be disposed on the upper surface of the membrane pad, and this will be described in detail through the operation process.

Meanwhile, the absorbent pad receptacle 514 may be formed to extend from the gripping part 51 to be integrally formed with the gripping part 51, but it may be formed separately to be able to be coupled to the gripping part 51 so as to be coupled to the upper plate 510 of the gripping part 51. Moreover, the sliding member 50a on which the absorbent pad receptacle 514 is formed may be applied to the diagnostic strip 1 in the first example and the diagnostic strip 1a in the second example.

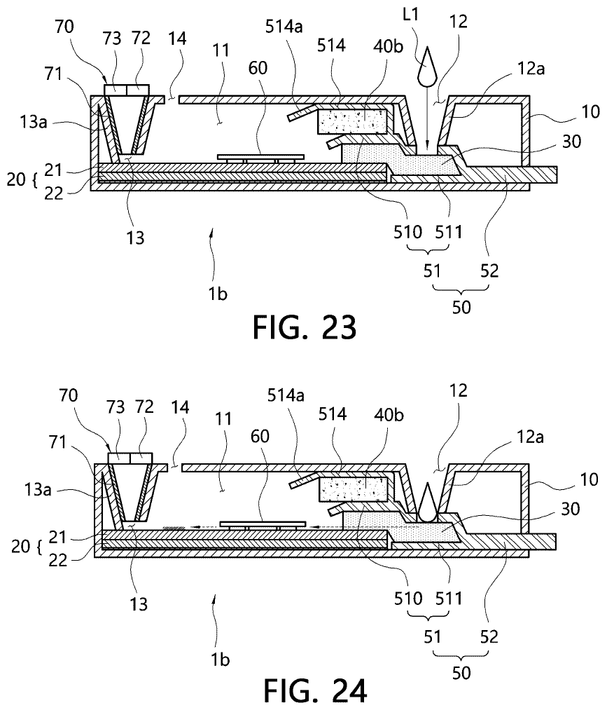

Hereinafter, the operation process of the diagnostic strip according to the third example of the present invention will be described with reference to FIGS. 23 to 27.

FIGS. 23 to 27 are operational diagrams illustrating the operation process of a diagnostic strip according to the third example of the present invention.

It is the same as the operation process of the first example described above, except that the shape of the sliding member 50a is deformed to slide to accommodate and discharge the absorbent pad 40b. Accordingly, the previously described operation process will be briefly described, and the operation process of the sliding member will be described in detail.

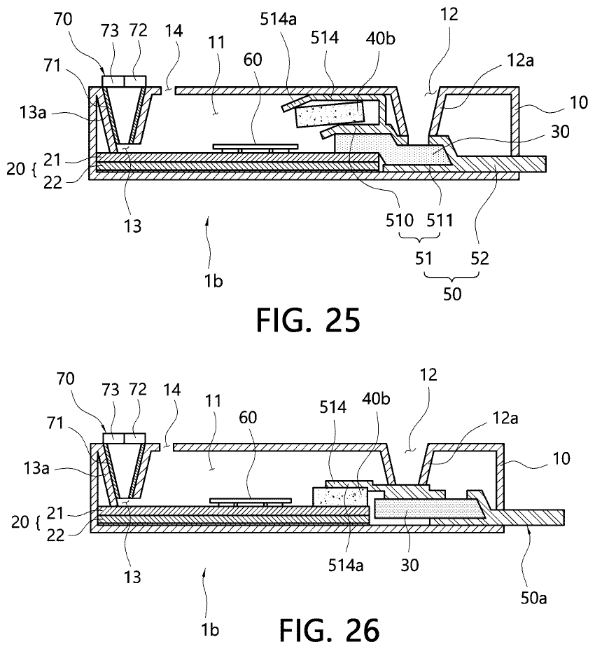

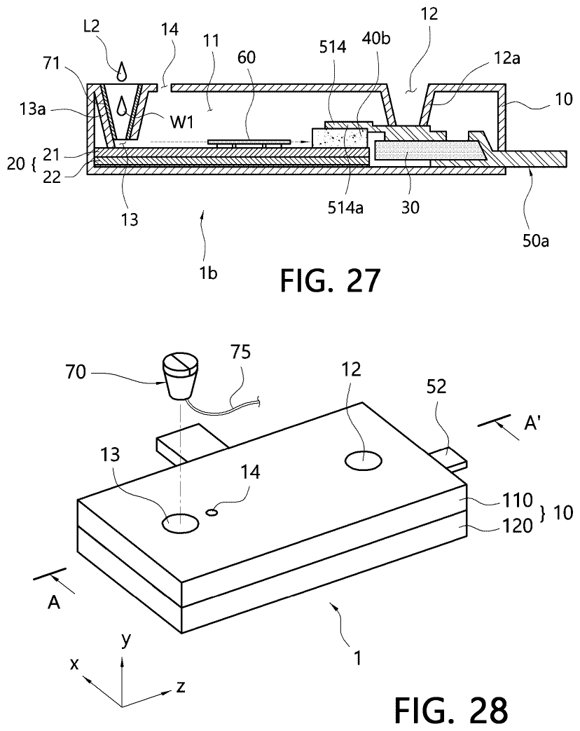

FIG. 23 is a diagram illustrating that a sample solution L1 is introduced into the first inlet 12, and FIG. 24 is a diagram illustrating that the sample pad 30 absorbs the sample solution L1 and is transferred to the membrane pad 20. FIG. 25 is a diagram illustrating that the sliding pad slides by pulling the extension part 52, and FIG. 26 is a diagram illustrating that the absorbent pad 40b is separated from the absorbent pad receptacle 514 to be in contact with the membrane pad 20, and FIG. 27 is a diagram illustrating that the washing solution W1 and the reaction solution L2 of the solution supply device 70 are introduced into the second inlet 13, and the absorbent pad 40b absorbs the same.

Referring to FIGS. 23 to 27, the sample solution L1 is introduced into the first inlet 12 from the outside of the housing 10. The sample solution L1 that is introduced into the first inlet 12 is absorbed by the sample pad 30, and the sample pad 30 comes into contact with the membrane pad 20 and transfer the sample solution L1 that is absorbed in the sample pad 30 to the membrane pad 20. When the absorption of the sample solution L1 from the upper portion of the housing 10 through the arrival confirmation port 14 to the lower side of the arrival confirmation port 14 is confirmed, the extension part 52 is pulled.

When the extension part 52 of the sliding member 50a is pulled from the outside of the housing 10, the gripping part 51 and the absorbent pad receptacle 514 may also slide in a direction in which the extension part 52 is pulled. The absorbent pad receptacle 514 may move downward of the first guide surface 12a, and it may be formed of a soft material such that the shape thereof is deformed to move by sliding. In this case, the absorbent pad 40b in which the absorbent pad receptacle 514 is accommodated inside by the first guide surface 12a may be discharged to the outside along the inclined surface 514a. As the sliding member 50 slides, the absorbent pad 40b may gradually protrude from the absorbent pad receptacle 514 and be seated on the upper surface of the membrane pad 20. In this case, the absorbent pad 40b may be disposed in close contact with the upper surface of the membrane pad 20 while pressing the absorbent pad 40b from the upper portion of the inclined surface 514a of the absorbent pad receptacle 514. In the diagnostic strip 1 according to the third example, the sliding member 50a includes the absorbent pad receptacle 514 and does not require a separate elastic member 41, and the absorbent pad 40b may be spaced apart from the upper surface of the membrane pad 20 or disposed to be in close contact with the upper surface of the membrane pad 20.

FIG. 27 is a cross-sectional diagram of a process in which the washing solution W1 and the reaction solution L2 are sequentially supplied, and the first and second receptacles 72, 73 are omitted for the convenience of description.

When the sliding member 50a slides to move the sample pad 30 away from the membrane pad 20 and the absorbent pad 40b is in close contact with the upper surface of the membrane pad 20, the washing solution W1 and the reaction solution L2 may be introduced through the second inlet 13. The washing solution that is introduced from the second inlet 13 is absorbed by the membrane pad 20 and transferred to the electrode part 60, and the antigen concentration may be measured by oxidation/reduction reaction.

The diagnostic strip 1 of the present invention may be applied to various sensors through an optical analysis method that analyzes discoloration, luminescence and fluorescence according to the selection of an enzyme bound to a detection antibody and a substrate according to the enzyme, and an electrochemical analysis method that analyzes an oxidation/reduction reaction

In addition, the diagnostic strip 1 of the present invention may be applied to the optical analysis and electrochemical analysis of discoloration, luminescence and fluorescence depending on the used enzyme and substrate.

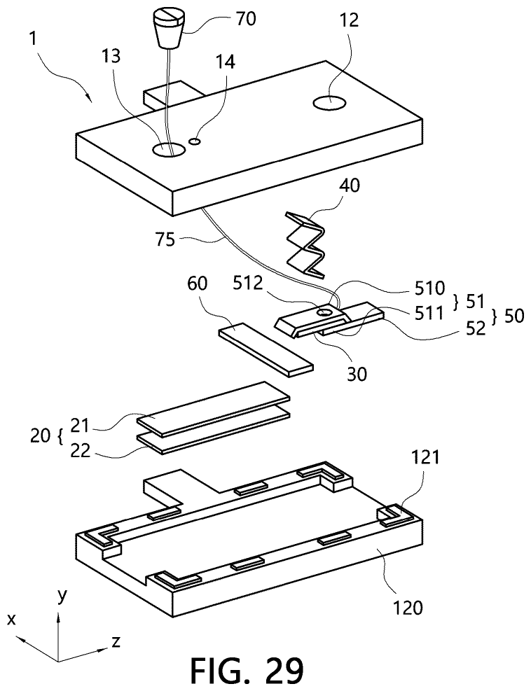

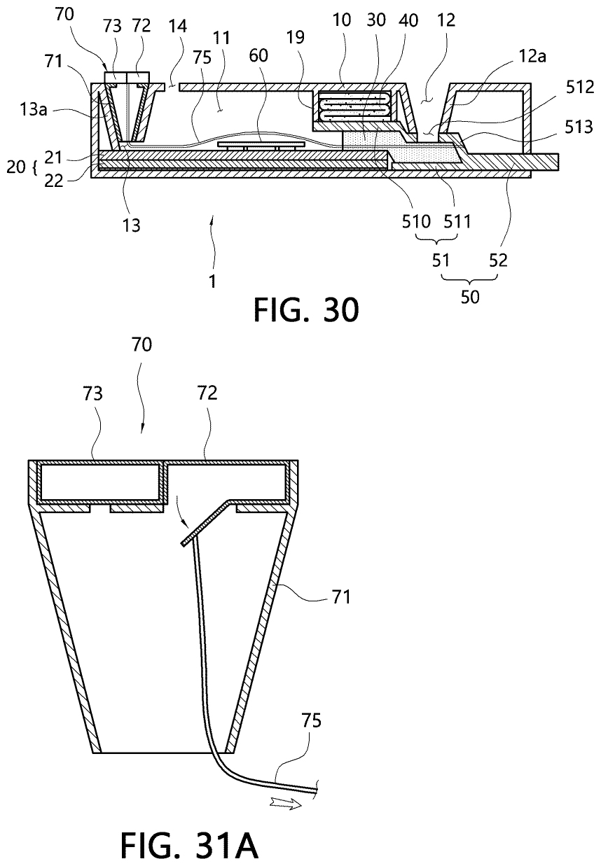

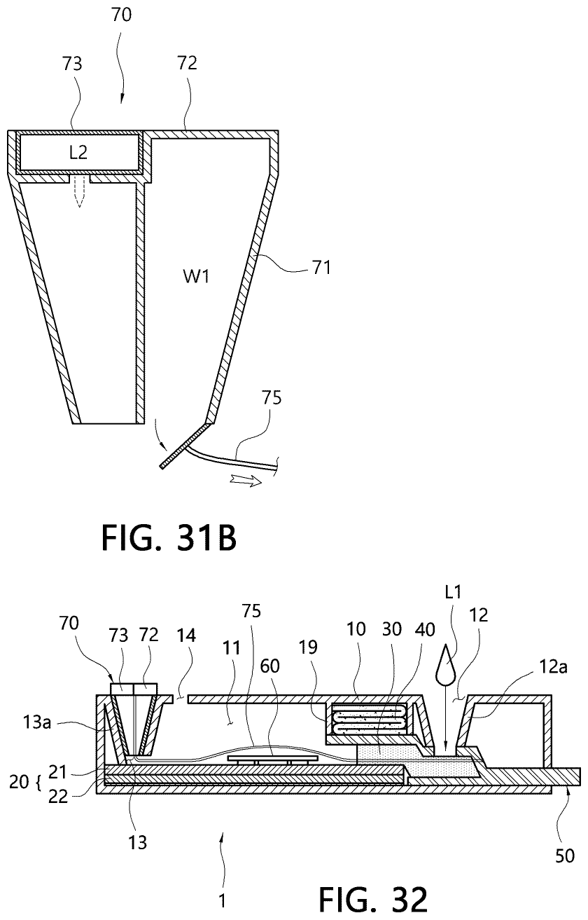

FIG. 28 is a perspective view of a diagnostic strip according to the fourth example of the present invention, FIG. 29 is an exploded perspective view of FIG. 28, FIG. 30 is a cross-sectional view taken along line A-A′ of the diagnostic strip of FIG. 28, and FIG. 31 is a perspective view of the automatic supply part.

Referring to FIGS. 28 to 31, respectively, the diagnostic strip 1 of the present invention includes a housing 10, a membrane pad 20, a sample pad 30, an absorbent pad 40 and a sliding member 50 that are positioned inside the housing 10.

Specifically, the diagnostic strip 1 of the present invention is formed with a receiving space 11 therein, and includes a housing 10 for forming a first inlet 12 and a second inlet 13 that are disposed to be spaced apart from each other by communicating the receiving space 11 and the external space on the upper surface, a membrane pad 20 which is exposed to the second inlet 13 and disposed in the receiving space 11, a sample pad 30 which is disposed in the receiving space 11 in contact with at least one surface of the membrane pad 20 and absorbs a sample solution L1 that is introduced through the first inlet 12, an absorbent pad 40 which is disposed between the first inlet 12 and the second inlet 13 in which one side surface thereof is in contact with the inner upper surface of the housing 10 and the other side surface thereof is spaced apart from the membrane pad 20, a gripping part 51 for gripping the sample pad 30, a sliding member 50 which includes an extension part 52 that is formed to extend from the gripping part 51 in a horizontal direction and at least a portion thereof penetrates the side surface of the housing to protrude outward, and an automatic supply part 70 which is integrally or detachably coupled to the second inlet 13 and supplies a washing solution W1 according to the movement of the sliding member, and also supplies a reaction solution L2 with a time difference from the supply of the washing solution W1, wherein the sliding member 50 slides in a horizontal direction such that the sample pad 30 is spaced apart from the membrane pad 20, and the other side surface of the absorbent pad 40 comes into contact with the membrane pad 20.

The housing 10 forms the main body of the diagnostic strip 1, and it may be formed in a rectangular parallelepiped shape to form a receiving space 11 therein. As illustrated in the drawings, it is described as an example that the housing 10 is formed to extend in the z-axis direction and a cross-section that is cut in a horizontal line is formed in a rectangular shape, but the present invention is not limited thereto, and a cross-sectional shape that is cut by a horizontal line may be formed in an elliptical shape rather than a rectangular shape. The housing 10 accommodates the membrane pad 20, the sample pad 30, the absorbent pad 40 and the sliding member 50 in the receiving space 11. The housing 10 may be separately formed vertically such that each component accommodated in the receiving space 11 can be easily arranged, and may include a first member 110 and a second member 120 that are separately formed to be able to be coupled. The first member 110 and the second member 120 form the upper and lower portions of the housing 10 that are separated vertically, respectively, and they may include an upper surface and a lower surface of the housing 10, respectively, and they may be separated and coupled to each other by coupling grooves (not illustrated) and protrusions 121 that are formed on surfaces facing each other. In the first member 110 including the upper surface of the housing 10, a first inlet 12 and a second inlet 13 communicating with the receiving space 11 and the outer space are formed to be spaced apart from each other, and an arrival confirmation port 14 may be formed between the first inlet 12 and the second inlet 13. The first inlet 12 and the second inlet 13 are through-holes for introducing the sample solution L1, the washing solution W1 and the reaction solution L2 into the receiving space 11, respectively, and they are formed through the upper and lower surfaces of the first member 110, and may be formed to be spaced apart along the z-axis, which is the longitudinal direction of the housing 10.

An automatic supply part 70 may be coupled or integrally fixed to the second inlet 13. The automatic supply part 70 includes a cylindrical housing 71 for coupling with the second inlet 13, a first receptacle 72 which is located at an upper part of the housing 71 to accommodate the washing solution W1, a second receptacle 73 which is located at the upper portion of the housing 71 and on the side surface of the first receptacle 72 to accommodate the reaction solution L2, and a strip line 75 that connects the strippable bottom surface of the first receptacle 72 and the sliding member 50 to open the bottom surface of the first receptacle 72 when the sliding member 50 moves.

As will be described below in detail, the bottom surface of the first receptacle 72 is opened according to the movement of the sliding member 50 to introduce the washing solution W1 into the second inlet 13, and the second receptacle 73 may inject the reaction solution L2, which is accommodated by a method such as breakage, breaking, cutting, pressing and the like, at an appropriate timing during the analysis process through the second inlet 13.

That is, by opening the bottom surface of the first receptacle 72 and injecting the washing solution W1 to proceed with the washing step, the second receptacle 73 is broken and the reaction solution L2 is injected to analyze the reactivity of HRP. In addition, the washing solution W1 and the reaction solution L2 may be injected into the second inlet in stages.

In the present specification, examples of the washing solution W1 may include a washing buffer that partially includes a surfactant such as tween and the like, and examples of the reaction solution L2 may include a solution including a substrate that reacts with an enzyme (peroxidase, phosphatase, etc.) which is bound to the detection antibody. The sample solution L1, the washing solution W1 and the reaction solution L2 may be introduced to be opposite to each other through the first inlet 12 and the second inlet 13. The first inlet 12 and the second inlet 13 have a feature that can minimize non-specific reactions by implementing the input directions in which the sample solution L1, the washing solution W1 and the reaction solution L2 are introduced in opposite directions. The arrival confirmation port 14 is a through-hole for confirming that the sample solution L1 that is introduced into the first inlet 12 has reached a specific position along the membrane pad 20, and it may be formed by penetrating the upper and lower surfaces of the first member, and it may be formed in parallel between the first inlet 12 and the second inlet 13 along the z-axis. The arrival confirmation port 14 may be formed to be spaced apart from the first inlet 12 and the second inlet 13, and be formed at a position adjacent to the second inlet 13 than the first inlet 12.

Meanwhile, the housing 10 may include a first guide surface 12a which extends from an inner upper surface, a second guide surface 13a and an indentation part 19.

When it is described in detail with reference to FIG. 30, the housing 10 may include a first guide surface 12a which is formed to extend downward from the lower surface of the first member 110, a second guide surface 13a, an indentation part 19 and a pressing member (not illustrated).

The first guide surface 12a and the second guide surface 13a are for guiding the movement of the sample solution L1, the washing solution W1 and the reaction solution L2 that are introduced into the first inlet 12 and the second inlet 13, and they may be formed to extend downward toward the receiving space 11 from the first inlet 12 and the second inlet 13. The first guide surface 12a and the second guide surface 13a may be formed to have a diameter of an end portion smaller than diameters of the first inlet 12 and the second inlet 13 to be inclined downward. The first guide surface 12a and the second guide surface 13a are formed to be inclined toward the lower portion so as to guide the sample solution L1, the washing solution W1 and the reaction solution L2 that are introduced into the first inlet 12 and the second inlet 13 to be introduced into specific positions of the sample pad 30 and the membrane pad 20. In addition, a portion of the first guide surface 12a is formed to have a longer length than other portions such that the membrane pad 20 can be closely fixed to the upper surface of the second member 120.

The indentation part 19 forms a space in which the absorbent pad 40 to be described below is inserted and guides the movement of the absorbent pad 40, and it may be formed to extend downward from the inner upper surface of the housing 10. The indentation part 19 may be formed to extend downward from the lower surface of the first member 110 between the first inlet 12 and the second inlet 13.

The indentation part 19 may be disposed to be closer to the first inlet 12 between the arrival confirmation port 14 and the first inlet 12 to form a space into which the absorbent pad 40 is inserted. The indentation part 19 accommodates the absorbent pad 40 in the inner space, and the opened lower side may be opened and closed by the sliding member 50, which will be described below in detail in the operation process.

The pressing member is for pressing the electrode part 60 which is disposed in the receiving space 11, and it may be formed to extend downward from the inner upper surface of the housing 10. The pressing member may be formed to extend downward from the lower surface of the first member 110 between the first inlet 12 and the second inlet 13. The pressing member may be formed at a position facing the electrode part 60 between the arrival confirmation port 14 and the indentation part 19 to press the electrode part 60.

The membrane pad 20 is disposed in the receiving space 11 of the housing 10 including the first member 110 and the second member 120.

The membrane pad 20 is disposed in the inner receiving space 11 of the housing 10.

The membrane pad 20 may be disposed on the upper surface of the second member 120 and may be disposed to be exposed by the second inlet 13. The membrane pad 20 may include a nitrocellulose (NC) pad 21, and may be formed to extend along the longitudinal direction of the housing 10 so as to face the second inlet 13, the arrival confirmation port 14, the pressing member and the indentation part 19. At least a portion of the membrane pad 20 is pre-treated with a capture antibody that specifically binds to a specific target antigen, and the detection antibody and capture material that binds secondarily to the antigen bound to the capture antibody are not limited to the antibody, and they may be composed of proteins, carbohydrates, nucleotides, aptamers and the like.

The membrane pad 20 may be formed by combining a cover which is capable of reducing the interaction between the fluid in the nitrocellulose pad 21 and the second member 120, and for example, it may be formed by combining the nitrocellulose pad 21 and polyethylene terephthalate 22 (PET). The membrane pad 20 may be formed of a member that minimizes interaction, such as a corrosion reaction by a fluid, and it may be a member having an adhesive formed thereon.

One end portion of the membrane pad 20 may absorb the sample solution L1 that is introduced from the first inlet 12 from the sample pad 30, and when the sample solution L1 moves in the longitudinal direction to reach the arrival confirmation port 14, it is possible to absorb the washing solution W1 and the reaction solution L2, which are introduced into the second inlet 13 from the other end portion. The reaction solution absorbed by the membrane pad 20 is transferred to the electrode part 60, and the electrode part 60 may generate an electric signal, which will be described in detail during the operation process. The membrane pad 20 is characterized in that one side surface is pre-treated with a capture antibody that specifically binds to a predetermined antigen. The membrane pad 20 is not limited to the detection antibody and the capture material that binds secondarily to the antigen bound to the capture antibody, and they may be composed of proteins, carbohydrates, nucleotides, aptamers and the like. The membrane pad 20 may be described as an example in which capture is coated in advance on one side surface, and the capture and detector may not be limited to materials of an antibody.

The membrane pad 20 may quantitatively analyze the reaction generated on the surface through the electrode part 60 by electrochemical analysis. Meanwhile, the electrode part 60 is disposed on the upper surface of the membrane pad 20, and the sample pad 30 that is in contact with and separated from the membrane pad 20 is disposed on one side of the membrane pad 20.

The electrode part 60 obtains a signal from a reaction in which the reaction solution that is introduced into the second inlet 13 and absorbed by the membrane pad is oxidized/reduced by the enzyme of the detection antibody, and it may be disposed to be in close contact with the upper surface of the membrane pad 20 that is located between the second inlet 13 and the absorbent pad 40. The electrode part 60 includes a board on which electrodes are patterned, and two electrodes or three electrodes may be patterned thereon. For example, the electrode part 60 includes a counter electrode, a working electrode and a reference electrode, and it may be disposed on the upper surface of the membrane pad 20 to face the pressing member. Such an electrode part 60 reacts with the solution that is introduced into the first inlet 12 and the second inlet 13 to cause an oxidation/reduction reaction to analyze the concentration or presence of an analyte that is included in the solution.

Meanwhile, the diagnostic strip 1 according to the fourth example of the present invention is described as an example of measuring the concentration of an antigen by oxidation/reduction reaction, but the present invention is not limited thereto, and the presence or concentration of an analyte may be analyzed by confirming that the membrane pad 40 is discolored without forming the electrode part 60. In order to analyze the signal of the presence or absence of binding of the detection antibody, the enzymes used in combination with the detection antibody are peroxidase and phosphatase, and typically, horseradish peroxidase and alkaline phosphatase may be used. As a substrate thereof, TMB, dianisidine, phenylenediamine, NBT/BCIP, pNPP and the like may be used for electrochemical and discoloration reactions, and lumino, CSPD, 1,2-dioxetane and the like may be used for luminescence reaction. In addition, fluorescence may be analyzed by attaching a fluorescent dye to the detection antibody.

The sample pad 30 is a pad that absorbs the sample solution L1 that is introduced into the first inlet 12. The sample pad 30 may be disposed under the first guide surface 12a so as to be exposed through the first inlet 12, and may be disposed to be in contact with at least one surface of the membrane pad 20 in the receiving space 11 so as to absorb the sample solution L1 that is introduced from the first inlet 12 to transfer to the membrane pad 20. As illustrated in FIG. 30, the lower surface of the sample pad 30 may contact the upper surface and one side surface of the membrane pad 20 at the same time, but the present invention is not limited thereto, and one side surface of the end portion may be in contact with one side surface of the end portion of the membrane pad 20, or it may be disposed in the receiving space 11 such that at least a portion thereof is in contact therewith. The sample pad 30 disposed in this way may be slidably moved in the horizontal direction, which is the longitudinal direction of the housing 10, by the sliding member 50, such that it can be contacted and separated from the membrane pad 20.

The sliding member 50 is for contacting and separating the sample pad 30 from the membrane pad 20, and it is formed of a soft material and disposed in the receiving space 11. The sliding pad includes a gripping part 51 for gripping the sample pad 30, and an extension part 52 which is formed to extend horizontally from the gripping part 51 such that at least a portion thereof penetrates the side surface of the housing 10 and is disposed to protrude to the outside, and the sample pad 30 may be in contact with and separated from the membrane pad 20 while sliding in the horizontal direction by pulling the extension part 52 which is disposed to protrude from the outside of the housing 10.

The gripping part 51 is for gripping the sample pad 30, and it may include an upper plate 510 and a lower plate 511 that are vertically spaced apart from each other. The gripping part 51 is formed in a ‘C’ shape to form a separation space in which the sample pad 30 is accommodated between the upper plate 510 and the lower plate 511, and it may form a through-hole 512 that penetrates the upper surface of the upper plate 510 to be in communication with the separation space and the first inlet 12. The gripping part 51 forms a through-hole 512 communicating with the first inlet 12 such that the sample pad 30 which is disposed in the separation space may absorb the sample solution L1 that is introduced into the first inlet 12. The gripping part 51 may form a bending part 513 in which an end portion of the upper plate 510 is bent downward. The bending part 513 is for preventing a phenomenon in which the sample pad 30 is separated from the separation space when the sliding member 50 slides.

The extension part 52 forms a handle for moving the gripping part 51, and it is formed top extend from the end of the gripping part 51 in the horizontal direction. The extension part 52 is formed to extend along the −z-axis direction from the gripping part 51, and the end portion penetrates the side surface of the housing 10 and is disposed to protrude outside the housing 10. At least a portion of the extension part 52 is disposed on the outside of the housing 10, and the user pulls the end portion of the extension part 52 to slide the sliding pad in the z-axis and −z-axis directions such that the sample pad 30 may be in contact with and separated from the membrane pad 20.

One end of the strip line 75 is connected to the sliding member 50, and when the sliding member 50 moves, the strip line 75 is pulled, and by stripping the lower surface of the first receptacle 72 to which the opposite end of the strip line 75 is connected, the received washing solution W1 flows out and is introduced through the second inlet 13 by gravity.

Through this process, after the sample solution L1 is introduced, the washing solution W1 may be automatically added simply by pulling the sliding member 50, and therefore, the washing solution W1 may be introduced at an appropriate time.

Meanwhile, although the sliding pad slides in the horizontal direction, it is described as an example that the extension part 52 is formed to extend in the z-axis and −z-axis directions and slides along the longitudinal direction of the housing 10, but the present invention is not limited thereto, and it may be formed to extend in the x-axis or −x-axis direction such that the sample pad 30 contacts with and is separated from the membrane pad 20 while sliding.

Subsequently, the gripping part 51 may be formed such that the upper plate 510 is longer than the lower plate 511. The gripping part 51 may be positioned below the indentation part 19 when the upper plate 510 is formed longer than the lower plate 511 and the sliding pad slides in the z-axis direction. The upper plate 510 of the gripping part 51 may be positioned below the indentation part 19 while sliding the sliding pad in the z-axis direction and the −z-axis direction, or may be positioned such that it is completely deviated from the lower side of the indentation part 19 so as to open or close the lower side of the indentation part 19. In this way, the sliding pad opens and closes the open lower side of the indentation part 19 such that the absorbent pad 40 accommodated in the indentation part 19 is completely accommodated inside the indentation part 19, or at least a portion thereof is separated from the indentation part 19 to come into contact with the membrane pad 20. This will be described below in detail through the operation process after the absorbent pad 40 is described.

The automatic supply part 70 of the present invention may be positioned only on the upper portion of the housing 71 in the form of a pouch as shown in FIG. 31a, and a portion of the housing 71 may be isolated to accommodate the washing solution W1 as shown in FIG. 4b.

The absorbent pad 40 is for absorbing the washing solution W1 that is introduced through the second inlet 13 and absorbed into the membrane pad 20, and it is formed of a material that expands in volume when the solution is absorbed. For example, the absorbent pad 40 may be formed of a material such as glass fiber, cotton or the like, and it may be disposed between the first inlet 12 and the second inlet 13. The thickness, material and volume of the absorbent pad 40 may be adjusted according to the amount of the washing solution W1 that is introduced through the second inlet 13, while the first receptacle 72 of the automatic supply part 70 is broken. Referring to FIG. 5a, the absorbent pad 40 may be formed of a thin pad which is capable of absorbing a solution. The absorbent pad 40 may be formed by being cut to a size that can be inserted into the indentation part 19, and the cut and formed thin pad may be formed by folding in different directions multiple times as illustrated in FIG. 5b. The absorbent pad 40 is described as an example that it is folded to form five layers as shown in the drawings, but the present invention is not limited thereto, and the area, volume, number of folds, thickness, material and the like of each layer may be adjusted according to the amount of the washing solution that is introduced into the second inlet 13.

The absorbent pad 40 may be folded multiple times in opposite directions to form a stacked structure, and one side surface thereof is in contact with the inner upper surface of the housing 10, and the other side surface thereof is disposed to be spaced apart from the membrane pad 20. The absorbent pad 40 may have different elasticity depending on the area, volume, thickness, number of times of folding, material and the like. The absorbent pad 40 may be stacked by applying pressure up and down after being folded multiple times as described in the other examples above, and if no pressure is applied to the upper or lower surfaces, each layer may be spaced apart and open up and down.

The absorbent pad 40 is inserted into the indentation part 19 such that the lower side of the indentation part 19 is closed by the sliding member 50 to be spaced apart from the membrane pad 20, and when the sliding member 50 slides and the lower side of the indentation part 19 is opened, a portion thereof may be separated from the indentation part 19 due to elasticity and may come into contact with the upper surface of the membrane pad 20 on the other side surface thereof.

That is, before the sliding member 50 slides, the gripping part 51 closes the lower side of the indentation part 19 such that the absorbent pad 40 is completely inserted into the indentation part 19, and thus, it is spaced apart from the membrane pad 20, and the sample pad 30 may be disposed to contact the membrane pad 20, and the through-hole 512 may be disposed to communicate with the first inlet 12.

In addition, when the extension part 52 of the sliding member 50 slides outwardly of the housing 10, the gripping part 51 opens the lower side of the indentation part 19 such that the absorbent pad 40 is in contact with the other side surface of the membrane pad 20, and the sample pad 30 moves away from the membrane pad 20. In addition, the upper surface of the gripping part 51 may close the end portion of the first guide surface 12a, and it will be described in detail with reference to the operation process.

FIGS. 32 to 36 are operational diagrams illustrating the operation process of a diagnostic strip according to the fourth example of the present invention.

Referring to FIG. 32, it illustrates a diagram for introducing a sample solution L1 into the first inlet 12. For the sample solution L1, a detection antibody-horseradish peroxide (HRP) solution that is pre-bound with the antigen sample to be analyzed is used. Peroxidase enzymes such as HRP can oxidize specific substrates, and the degree of such an enzyme-substrate oxidation/reduction reaction can quantitatively measure the amount of antigen through an optical measurement method such as discoloration, luminescence, fluorescence analysis and the like, as well as a highly sensitive electrochemical measurement method. As such, the sample solution L1 in which the detection antibody and HRP are combined is introduced into the first inlet 12. In this case, the sample pad 30 must be exposed through the first inlet 12, and the sample solution L1 must be introduced into the first inlet 12 while the sample pad 30 is in contact with the membrane pad 20. The sample solution L1 that is introduced into the first inlet 12 may be absorbed into the sample pad 30.

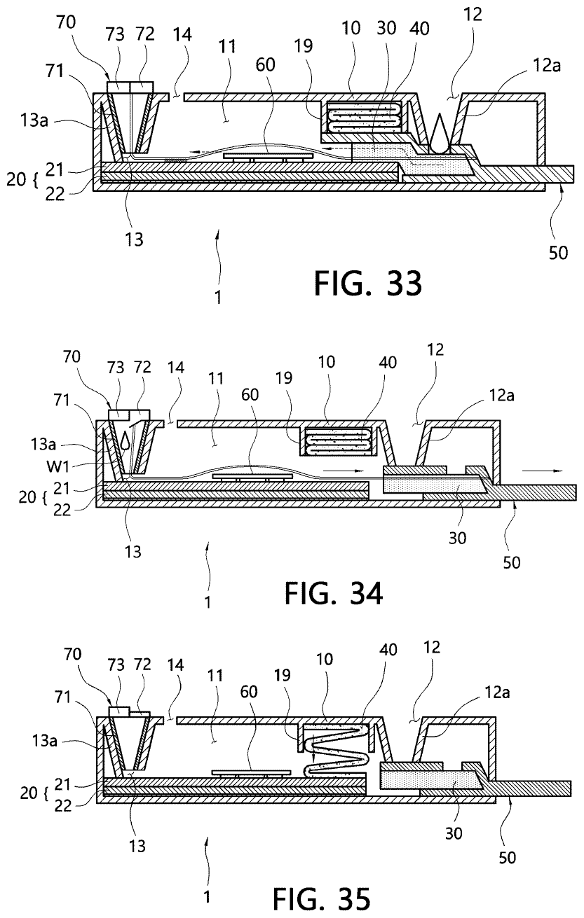

Referring to FIG. 33, it illustrates a diagram in which the sample solution L1 that is introduced into the first inlet 12 falls on the sample pad 30, and the sample pad absorbs the sample solution L1 such that the membrane pad 20 which is in contact with the sample pad 30 becomes wet.

The sample pad 30 absorbs the sample solution L1 that is introduced from the first inlet 12, and at least a portion thereof is in contact with the membrane pad 20 such that the absorbed sample solution L1 is transferred to the membrane pad 20. The membrane pad 20 can absorb the sample solution L1 from one end portion which is in contact with the sample pad 30, and the sample solution L1 that is absorbed from the sample pad 30 becomes wet from one end portion to the other end portion. It can be confirmed that the sample solution L1 becomes wet from one end portion of the membrane pad 20 through the sample pad 30 to the other end portion through the electrode part 60. In this case, it can be confirmed that the sample solution L1 is absorbed from the arrival confirmation port 14 to the other end portion of the membrane pad 20, and when the sample solution L1 reaches the lower side portion of the arrival confirmation port 14, the sample pad 30 is separated from the membrane pad 20.

Referring to FIG. 34, it illustrates a diagram of sliding the sliding pad such that the sample pad 30 and the membrane pad 20 are separated. When the sample solution L1 is absorbed by the membrane pad 20 and reaches the lower side of the arrival confirmation port 14, the extension part 52 of the sliding pad is pulled from the outside of the housing 10. When the extension part 52 is pulled, the gripping part 51 which is integrally formed with the extension part 52 may slide and move, and at the same time, the sample pad 30 is separated from the membrane pad 20. When the gripping part 51 slides while the sliding pad is pulled to the outside of the housing 10, the sample pad 30 is separated from the membrane pad 20, and the upper plate 510 of the gripping part 51 may open the lower side of the indentation part 19. As the lower side of the indentation part 19 is opened, the absorbent pad 40 which is inserted and disposed in the indentation part 19 may at least partially protrude from the indentation part 19 by elasticity.

In this case, as the strip line 75 is pulled according to the movement of the sliding member 50, the lower surface of the first receptacle 72 accommodating the washing solution W1 is stripped, and the accommodated washing solution W1 is dropped to be introduced into the inside through the second inlet 13.

The washing solution W1 flows into the receiving space 11 through the second inlet 13, falls on the upper surface of the membrane pad 20, and is absorbed along the longitudinal direction of the membrane pad 20 to be in contact with the electrode part 60, and the remainder may be absorbed by the absorbent pad 40.

Referring to FIG. 35, it illustrates a diagram in which a portion of the absorbent pad 40 protrudes to the outside of the indentation part 19 due to elasticity. When the lower side of the indentation part 19 is opened, the absorbent pad 40 may protrude to the outside of the indentation part 19 by elasticity, and the other end portion may contact the upper surface of the membrane pad 20. One end portion of the absorbent pad 40 may be in contact with the inner upper surface of the indentation part 19 due to elasticity, and the other end portion may be disposed so as to be in contact with the upper surface of the membrane pad 20. In this case, one end portion of the absorbent pad 40 may be fixed to the inner upper surface of the indentation part 19, but it may not be fixed and may be in close contact by applying a force to the inner upper surface of the indentation part 19 by elasticity.

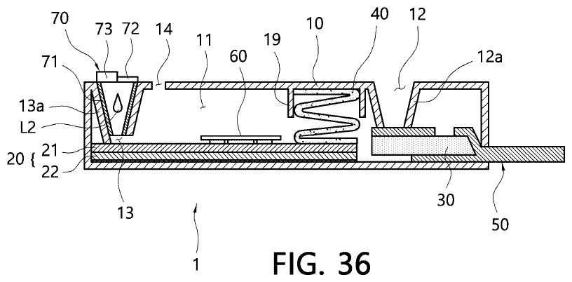

Subsequently, referring to FIG. 36, it illustrates a diagram in which the reaction solution L2 is introduced into the second inlet 13. While the membrane pad is in contact with the absorbent pad 40 and separated from the sample pad 30, the second receptacle 73 is broken, and the reaction solution L2 accommodated in the second receptacle 73 may be introduced into the second inlet 13.

The material of the second receptacle 73 may be vinyl or a resin material, and the reaction solution L2 accommodated therein is discharged by manually or automatically applying pressure or breaking part or all of the bottom surface so as to be introduced through the second inlet 13.

Examples of breaking the second receptacle 73 will be described below in more detail.

The electrode part 60 may induce an electrochemical reaction of the reaction solution L2. TMB is oxidized by the HRP enzyme to generate TMB radicals, hydrogen ions and electrons, and the process of reducing TMB that is oxidized according to Formula (2) by an electrochemical reaction at the working electrode is cyclically repeated. By using this principle, the current difference at which the maximum instantaneous current for the oxidation of TMB occurs can be confirmed by cyclic voltammetry, and the antigen concentration can be measured by the amperometric method. Accordingly, there is an advantage in that an amplified signal can be obtained even with a low concentration of antigen while the oxidation reaction by the enzyme and the reduction reaction by the electrode are continuously cycled.

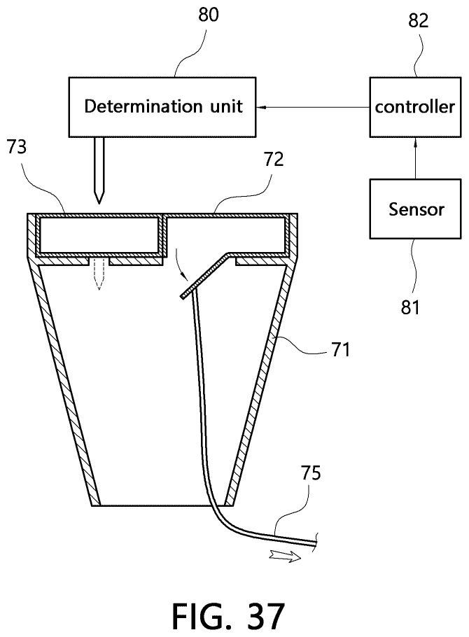

FIG. 37 is an exemplary diagram of the configuration for breaking the second receptacle 73 of the automatic supply part 70 of the present invention.

Referring to FIG. 37, a breaking part 80 for breaking the second receptacle 73 of the automatic supply part 70 may be provided. The breaking part 80 is operated by a sensor 81 for detecting the position of the extension part 52, and a controller 82 for outputting an operation control signal, after a set time has elapsed after the sensor 81 detects the movement of the extension part 52, so as to break the second receptacle 73. That is, after a predetermined time has elapsed after the bottom surface of the first receptacle 72 is stripped, the second receptacle 73 is broken.

In this case, the breaking part 80 may be installed on the diagnostic strip, provided on an analysis device using the diagnostic strip, or separately provided outside.

The breaking part 80 includes an awl 83, and by the movement of the awl, the second receptacle 73 is punctured, and the reaction solution L2 flows into the second receptacle 73 by gravity such that it may be introduced into the lower second inlet 13.

In the above example, the breaking part 80 for forming a hole in the second receptacle 73 by using the awl 83 has been described, but examples of breaking the second receptacle 73 may be implemented in various ways. The structure having the breaking line 74 described above with reference to FIG. 13 may be used.

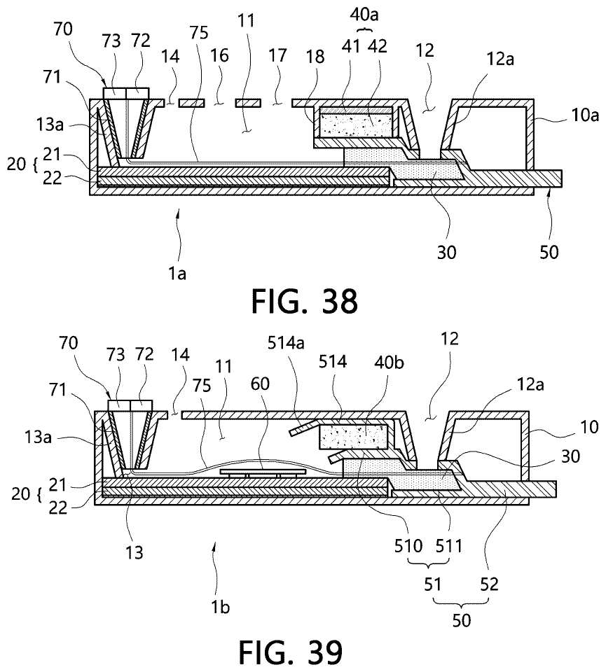

FIG. 38 is a cross-sectional configuration diagram of the diagnostic strip according to the fifth example of the present invention.

The configuration illustrated in FIG. 38 is that the automatic supply part 70 of the fourth example is applied to the diagnostic strip structure of the second example described with reference to FIGS. 15 to 20 of described above, and the detailed descriptions thereof will be omitted.

In addition, FIG. 39 is a cross-sectional configuration diagram of a diagnostic strip according to the sixth example of the present invention.

The diagnostic strip according to the sixth example of the present invention is an example in which the automatic supply part 70 of the fourth example is applied to the diagnostic strip structure of the third example.

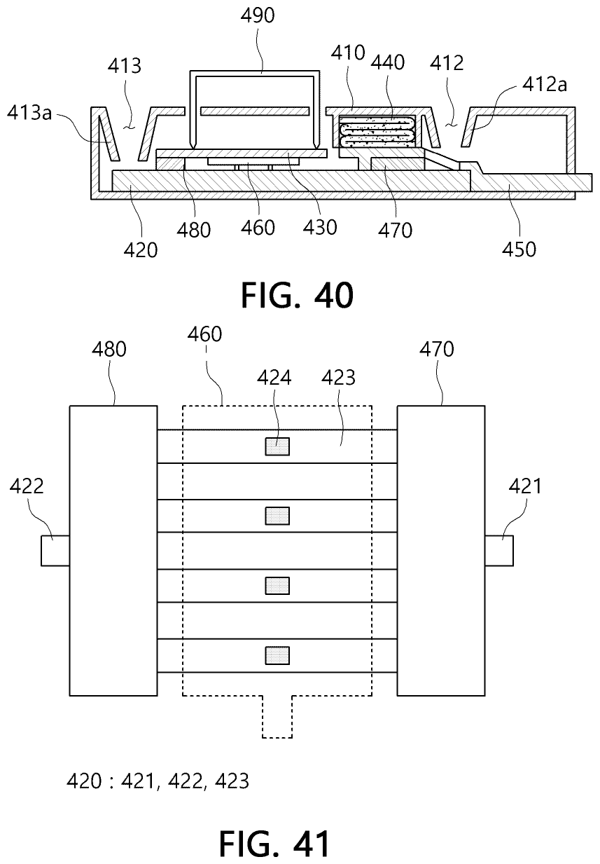

FIG. 40 is a cross-sectional configuration view of a diagnostic cartridge according to the seventh example of the present invention, and FIG. 41 is a plan view of major parts of FIG. 40.

Referring to FIGS. 40 and 41, respectively, the diagnostic cartridge of the present invention is for general users to conveniently measure the concentration or presence of an analyte which is present in bodily fluids such as blood and urine, and to quickly and accurately analyze in the field.

The diagnostic strip of the present invention includes a housing 410, a membrane pad 420, an absorbent pad 440, a first space forming part 470, a second space forming part 480, a liquid impermeable sheet 430 and a sliding member 450 that are positioned inside the housing 410.

The housing 410 forms a receiving space 411 therein, and includes a first inlet 412 and a second inlet 413 that are disposed to be spaced apart from each other by communicating with the receiving space 411 and the outer space on the upper surface.

In addition, it includes a membrane pad 420 which is exposed through the first inlet 412 and the second inlet 413 and disposed in the receiving space 411, an absorbent pad 440 which is disposed between the first inlet 412 and the second inlet 413 and in which one side surface is in contact with the inner upper surface of the housing 410 and the other side surface is spaced apart from the membrane pad 420, a sliding member 450 in which a portion thereof penetrates the side surface of the housing 410 to protrude outward, a first space forming part 470 for diffusing the sample solution that is introduced through the first inlet 412 in the membrane pad 420, a second space forming part 480 for diffusing the washing solution and the reaction solution that are introduced through the second inlet 413, a liquid impermeable sheet 430 which is disposed in the form of surrounding the upper and side surfaces of the membrane pad 420 to prevent the evaporation of a diffusing solution, and a cutting part for cutting the membrane pad 420 on both sides of the lower portion of an electrode part 460 before performing the measurement through the electrode part 460 after the reaction is completed.

The sliding member 450 slides in a horizontal direction to support and expose the bottom surface of the absorbent pad 440, and separates the first space forming part 470 which is positioned above the membrane pad 420 such that the other side surface of the absorbent pad 440 comes into contact with the membrane pad 420.

For diagnosis in such a structure, a sample solution is introduced through the first inlet 412, and after a predetermined time elapses, the washing solution and the reaction solution are sequentially introduced through the second inlet 413, respectively.

The sample solution which is introduced through the first inlet 412 may be urine or blood, the washing solution is a solution that can be washed by partially including a surfactant, and the reaction solution may be a solution including a substrate that reacts with an effect bound to the detection antibody.

The reason why the first inlet 412 and the second inlet 413 are positioned in opposite directions, and the sample solution, washing solution and reaction solution are introduced in opposite directions can be understood to minimize non-specific reactions.

The sample solution that is introduced through the first inlet 412 moves downward along the guide surface 412a of the first inlet 412, and it is introduced on one end of the upper surface of the membrane pad 420.

The membrane pad 420 may include a first pad 421 and a second pad 422 which protrude from both ends and are positioned below the first inlet 412 and the second inlet 413, a diagnostic line 423 which connects between the first pad 421 and the second pad 422 and is branched to form a plurality of diffusion paths, and a reaction part 424 which is located in the center of the diagnosis line 423

The membrane pad 420 may be made of nitrocellulose (NC) and, if necessary, it may be formed of a member that minimizes interaction such as a corrosion reaction by a fluid, and the lower surface may be attached to the bottom surface of the housing 410 by an adhesive.

The sample solution that is introduced through the first inlet 412 is supplied to the first pad 421.

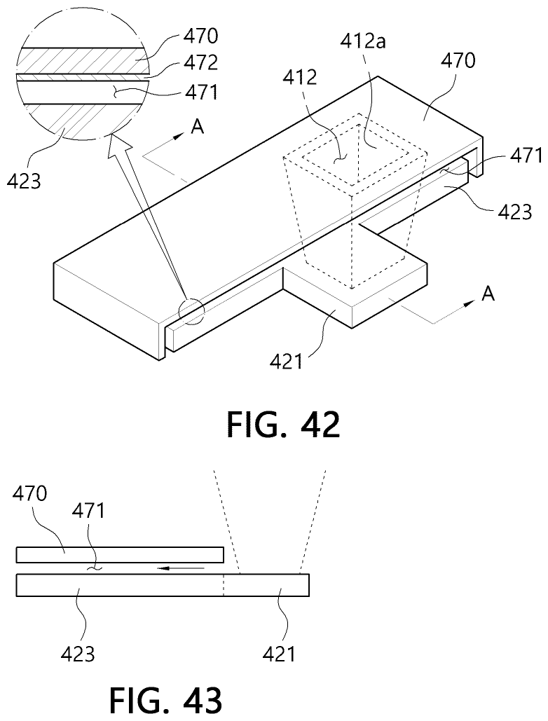

FIG. 42 is a partial perspective view of the present invention, and FIG. 43 is a cross-sectional view taken along line A-A in FIG. 42.

Referring to FIGS. 42 and 43, respectively, the sample solution which is supplied to the first pad 421 is absorbed and diffused into the membrane pad 420, and a microcavity 471, which is a minute interval, is formed between the membrane pad 420 and the first space forming part 470 by the first space forming part 470 that are positioned at a predetermined distance from the upper and side portions of the diagnostic lines 423 adjacent to the first pad 421.

The microcavity 471 causes a capillary phenomenon to diffuse the sample solution from the first pad 421 toward the reaction part 424.

A surface of the first space forming part 470 facing the membrane pad 420 may have a hydrophilic coating layer 472 formed thereon in order to generate a capillary phenomenon in the microcavity 471.

Therefore, it is possible to uniformly diffuse the sample solution regardless of the dispensing distance between the plurality of diagnostic lines 423.

In addition, since a liquid impermeable sheet 430 is positioned around the periphery including all or part of the upper portion of the diagnosis line 423 of the membrane pad 420, it prevents the evaporation of a sample solution to enable more accurate diagnosis.

Next, after the sample solution is diffused to the reaction part 424 and the position is confirmed through an arrival confirmation port (not illustrated), the sliding member 450 is slidably moved.

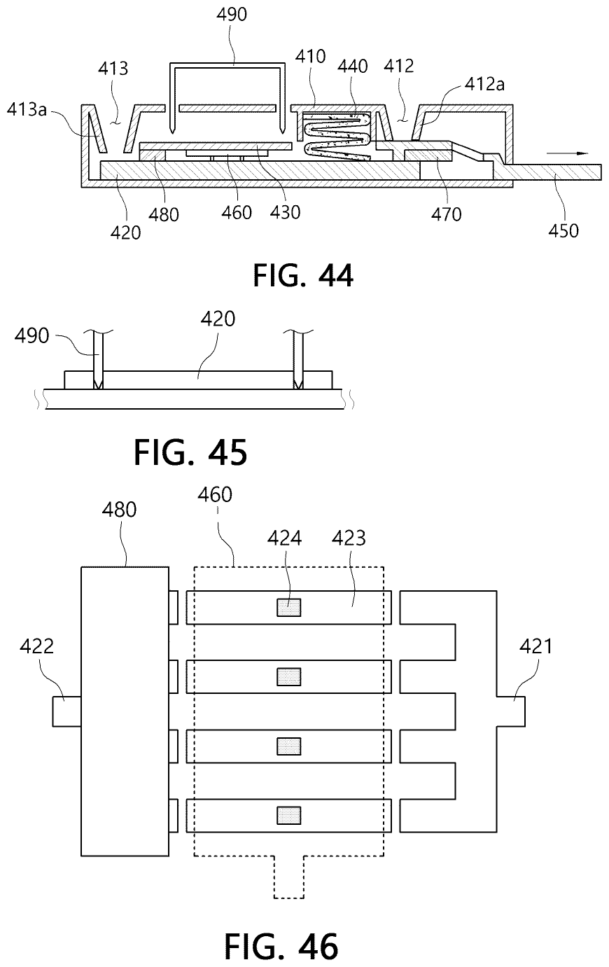

FIG. 44 is a cross-sectional view of a state in which the sliding member 450 is moved.

As the sliding member 450 moves, the bottom surface of the absorption pad 440 is exposed while the bottom surface of the absorption pad 440 is blocked.

Accordingly, part or all of the absorbent pad 440 moves downward.

In this case, as the sliding member 450 moves, the first space forming part 470 is also separated from the upper portion of the membrane pad 420, and the absorbent pad 440 comes into contact with the exposed membrane pad 420.

Next, the washing solution is introduced through the second inlet 413.

The washing solution moves downward along the second guide surface 413a, which is a side surface of the second inlet 413, and it falls on the second pad 422 and is absorbed in FIG. 41. In this case, the second space forming part 480 is positioned on a portion of the diagnostic line 423 which is adjacent to the second pad 422 such that the supplied washing solution can be rapidly diffused toward the reaction part 424.

The structure and operation in this case can be easily understood by using the examples of FIGS. 42 and 43 described above.

As mentioned above, the washing solution includes a surfactant, and it serves to remove residues and is absorbed by the absorbent pad 440.

Next, the reaction solution is again introduced through the second inlet 413.

The introduced reaction solution is absorbed toward the second pad 422, and in this case, the reaction solution is diffused toward the reaction part 424 by the microcavity formed by the second space forming part 480.

Accordingly, the washing solution and the reaction solution may be uniformly diffused regardless of the difference in dispensing distances due to the formation of a plurality of diagnostic lines 423.

Next, after the reaction between the reaction solution and the sample solution occurs, a diagnosis is made through the electrode part 460.

Before such diagnosis, it is necessary to remove unnecessary elements for a more accurate diagnosis. In particular, the present invention has a plurality of diagnostic lines 423 and a reaction part 424 in the center of each diagnostic line 423, and it is necessary to process each diagnostic line 423 such that it can be independently diagnosed.

To this end, as illustrated in FIG. 45, the user presses the cutting part 490 and moves it downward to cut the diagnostic line 423 on both ends of the electrode part 460.

The cutting part 490 is preferably formed integrally with the cartridge in order to prevent cross-contamination, and it may be supported by an elastic member to return to the original position after cutting.

The planar shape of the membrane pad 420 which is cut by the action of the cutting part 490 is illustrated in FIG. 46.

As described above, according to the present invention, a plurality of diagnostic lines 423 may be separated independently from each other by the action of the cutting part 490, and a reaction in the reaction part 424 which is located in each diagnostic line 423 may be detected and diagnosed.

The electrode part 460 obtains a signal from a reaction in which a reaction solution that is introduced into the second inlet 413 and absorbed by the membrane pad 420 is oxidized/reduced by the enzyme of a detection antibody, and it may be disposed in close contact with the upper surface of the membrane pad 420 which is positioned between the second inlet 413 and the absorbent pad 440.

The electrode part 460 includes a board on which electrodes are patterned, and two electrodes or three electrodes may be patterned thereon. For example, the electrode part 460 includes a counter electrode, a working electrode and a reference electrode, and it may be disposed on the upper surface of the membrane pad 20 to face the pressing member. Such an electrode part 460 reacts with the solution that is introduced into the first inlet 412 and the second inlet 413 to cause an oxidation/reduction reaction to analyze the concentration or presence of an analyte that is included in the solution.

Meanwhile, the present invention is described as an example of measuring the concentration of an antigen by oxidation/reduction reaction, but the present invention is not limited thereto, and the presence or concentration of an analyte may be analyzed by confirming that the membrane pad 440 is discolored without forming the electrode part 460. In order to analyze the signal of the presence or absence of binding of the detection antibody, the enzymes used in combination with the detection antibody are peroxidase and phosphatase, and typically, horseradish peroxidase and alkaline phosphatase may be used. As a substrate thereof, TMB, dianisidine, phenylenediamine, NBT/BCIP, pNPP and the like may be used for electrochemical and discoloration reactions, and lumino, CSPD, 1,2-dioxetane and the like may be used for luminescence reaction. In addition, fluorescence may be analyzed by attaching a fluorescent dye to the detection antibody.

Particularly, in the present invention, it is assumed that each of a plurality of branched diagnostic lines 423 connecting between the first pad 421 and the second pad 422 may be diagnosed.

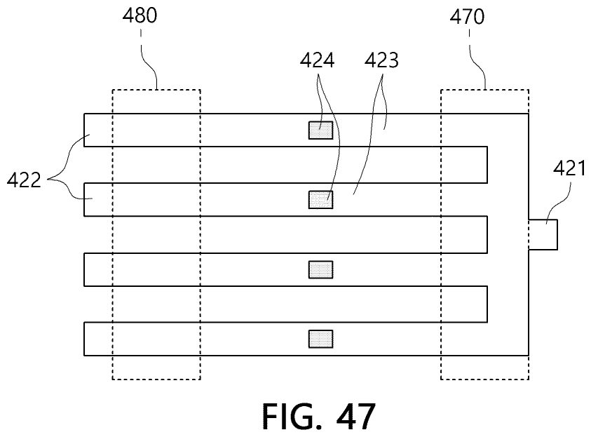

FIG. 47 is a configuration diagram of the membrane pad 420 according to another exemplary embodiment of the present invention.

Referring to FIG. 8, the membrane pad 420 may include a plurality of second pads 422, and it is respectively connected to a single first pad 421 by a diagnostic line 423.

The housing 410 for using the membrane pad 420 having this structure must have the same number of second inlets 413 to expose the plurality of second pads 422, respectively.

A sample solution is introduced into the single first pad 421, and it is absorbed and diffused to each side of the second pad 422.

After the washing solution is introduced through each of the plurality of second inlets 413, different reaction solutions are again introduced into each of the second inlets 413, and thus, different reactions may occur for each reaction part 424.

Thereafter, after each diagnosis line 423 is cut by using the cutting part 490, diagnosis results for different reactions may be obtained through the electrode part 460, respectively.

In the above, another example of the present invention has been described with reference to FIGS. 40 to 47. In the description with reference to FIGS. 40 to 47, the description related to the automatic injection of the solution has been omitted, but applying the automatic solution introduction of other previous examples to the example described with reference to FIGS. 40 to 47 can be easily performed by a person having ordinary skill in the art to which the present invention pertains.

Although the exemplary embodiments of the present invention have been described above with reference to the accompanying drawings, those of ordinary skill in the art to which the present invention pertains will understand that the present invention can be implemented in other specific forms without changing the technical spirit or essential features thereof. Therefore, it should be understood that the exemplary embodiments described above are illustrative in all respects and not restrictive.

### INDUSTRIAL APPLICABILITY

The present invention has improved the usability and performance of a diagnostic kit by applying mechanical and chemical constitutions by using natural laws, and thus has industrial applicability.

1-20. (canceled)

21. A diagnostic cartridge, comprising:
a housing that has formed therein a receiving space, and has formed, on the upper surface thereof, a first inlet and a second inlet that are spaced apart from each other in communication with the receiving space and an outer space; and
a solution supply device that is integrally or detachably mounted in the second inlet, receives a washing solution and a reaction solution, and sequentially introduces at intervals the washing solution and the reaction solution into a membrane pad through the second inlet.

22. The diagnostic cartridge of claim 21, wherein the solution supply device is an automatic supply part which is mounted integrally or detachably to the second inlet, receives a washing solution and a reaction solution and introduces the washing solution into the membrane pad when a sliding member moves, and after a predetermined time has elapsed after the washing solution is introduced, the reaction solution is introduced into the second inlet.

23. The diagnostic cartridge of claim 22, wherein the automatic supply part comprises:
a first housing which is coupled to the second inlet;
a first receptacle which is located at an upper part of the first housing and receives the washing solution, and in which the bottom surface is removed by a strip line having one end connected to the bottom surface and the other end connected to the sliding member so as to introduce the washing solution into the second inlet; and
a second receptacle which is located at an upper part of the first housing and a side surface of the first receptacle to receive the reaction solution, and is broken by the action of a breaking part to introduce the washing solution into the second inlet.

24. The diagnostic cartridge of claim 23, wherein the breaking part is operated after the sliding member slides and a set time has elapsed, and breaks by puncturing or pressing the second receptacle.

25. The diagnostic cartridge of claim 21, wherein the solution supply device comprises:
a first housing which is coupled to the second inlet;
a first receptacle which is located at an upper part of the first housing, receives the washing solution, and is broken by the action of a breaking part so as to introduce the washing solution into the second inlet; and
a second receptacle which is located at an upper part of the first housing and a side surface of the first receptacle to receive the reaction solution, and is broken by the action of the breaking part to introduce the washing solution into the second inlet.

26. The diagnostic cartridge of claim 25, wherein the breaking part breaks by sequentially puncturing or pressing the first receptacle and the second receptacle.

27. The diagnostic cartridge of claim 24, wherein breaking lines are formed on the bottom surfaces of the first receptacle and the second receptacle such that the breaking lines are broken by the pressure of the breaking part.

28. The diagnostic cartridge of claim 21, wherein the membrane pad is pre-treated with a capture antibody that specifically binds to a predetermined antigen on one side surface.

29. The diagnostic cartridge of claim 21, further comprising:
an absorbent pad which is disposed in the receiving space between the first inlet and the second inlet and has one side surface in contact with the inner upper surface of the housing and the other side surface spaced apart from the membrane pad,
wherein the absorbent pad comprises an elastic member which is in contact with an inner upper surface of the housing and an absorbent member which is disposed below the elastic member.

30. The diagnostic cartridge of claim 21, further comprising:
an absorbent pad which is disposed in the receiving space between the first inlet and the second inlet and has one side surface in contact with the inner upper surface of the housing and the other side surface spaced apart from the membrane pad,
wherein the absorbent pad is formed by being folded multiple times in different and opposite directions, and
wherein the area of each layer, the number of folds and the volume of the absorbent pad are adjusted according to the amounts of the washing solution and the reaction solution introduced into the second inlet.

31. The diagnostic cartridge of claim 30, further comprising:
a sample pad which is disposed in the receiving space to be in contact with at least one surface of the membrane pad and absorbs the sample solution introduced through the first inlet; and
a sliding member which comprises a gripping part for gripping the sample pad and an extension part which is formed to extend in a horizontal direction from the gripping part and has at least a portion protruding outward through a side surface of the housing,
wherein the sliding member slides in a horizontal direction such that the sample pad is spaced apart from the membrane pad, and the other side surface of the absorbent pad is in contact with the membrane pad, and
wherein the gripping part comprises an upper plate and a lower plate that are spaced apart vertically to form a separation space in which the sample pad is accommodated, and comprises a through-hole which penetrates an upper surface of the upper plate and communicates with the sample pad and the first inlet, and the gripping part forms a bending part in which both ends of the upper plate are bent toward a lower part.

32. The diagnostic cartridge of claim 31, wherein the gripping part comprises an absorbent pad receptacle which extends upward from an upper surface of the upper plate and has the other end bent in a direction opposite to the extension part to accommodate the absorbent pad therein.

33. The diagnostic cartridge of claim 32, wherein the absorbent pad receptacle forms an inclined surface with an end portion inclined toward the lower part, and
wherein the absorbent pad moves along the inclined surface when the sliding member slides and is seated on the upper surface of the membrane pad.

34. The diagnostic cartridge of claim 32, further comprising:
an indentation part which is formed to extend from the inner upper surface of the housing to the lower part between the first inlet and the second inlet to form a space into which the absorbent pad is inserted, and in which the lower side can be opened and closed by the sliding member;
an electrode part which is located between the second inlet and the absorbent pad and is closely disposed on the upper surface of the membrane pad; and
a pressing member which is formed to extend downward from the inner upper surface of the housing to face the electrode part and presses an electrochemical sensor unit.

35. A diagnostic cartridge, comprising:
the housing of claim 21;
a membrane pad which is disposed in the receiving space such that both ends of the housing are exposed to a first inlet and a second inlet, and comprises a plurality of diagnostic lines; and
a cutting part which is installed in the housing and cuts such that a plurality of the diagnostic lines are divided.

36. The diagnostic cartridge of claim 35, wherein the membrane pad comprises:
a first pad which is exposed to the outside through the first inlet;
a second pad which is exposed to the outside through the second inlet;
a plurality of diagnostic lines for connecting the first pad and the second pad; and
reaction parts that are located in each of the diagnostic lines.

37. The diagnostic cartridge of claim 36, wherein the second pad is provided in plurality, and
wherein the second pad is connected to the first pad by the same number of diagnostic lines as the second pad.

38. The diagnostic cartridge of claim 36, further comprising:
a first space forming part and a second space forming part for forming a microcavity between the diagnostic lines at the upper part and the side surface of the diagnostic lines that are adjacent to the first pad and the second pad.

39. The diagnostic cartridge of claim 38, wherein each of the first space forming part and the second space forming part has a hydrophilic coating layer formed on a surface facing the diagnosis line.

40. The diagnostic cartridge of claim 38, further comprising:
a liquid impermeable sheet which covers part or all of the diagnostic line.
